SEQUENCE LISTING (11 sequences)

SEQ ID NO: 1 - DNA, 30 bp, Artificial Sequence
  Primer
gatctttttattaaaaatataaaaattagt

SEQ ID NO: 2 - DNA, 23 bp, Artificial Sequence
  Primer
gatcccaaactaaaatacaataa

SEQ ID NO: 3 - DNA, 46 bp, Artificial Sequence
  Primer
gggacaccgctgatcgtatatttttattaaaaatataaaaattagt

SEQ ID NO: 4 - DNA, 19 bp, Artificial Sequence
  Primer
ccaaactaaaatacaataa

SEQ ID NO: 5 - DNA, 20 bp, Artificial Sequence
  Primer
gggacaccgctgatcgtata

SEQ ID NO: 6 - DNA, 19 bp, Artificial Sequence
  Primer
aataactaaaattacaaac

SEQ ID NO: 7 - DNA, 25 bp, Artificial Sequence
  Primer
ttgagttgtggtgggttttatttag

SEQ ID NO: 8 - DNA, 25 bp, Artificial Sequence
  Primer
tcatctcactaaaaaataccaaaca

SEQ ID NO: 9 - DNA, 1147 bp, Human
gggggaggagccaagatggccgaataggaacagctccggtctacagctcccagcgtgagcgacgcagaagacgggtgatttctgcatttccatctgaggtaccgggttcatctcactagggagtgccagacagtgggcgcaggccactgtgtgcgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcagagggtcctacgcccacggaatctcgctgattgctagcacagcagtctgagatcaaactgcaaggcggcaacgaggctgggggaggggcgcccgccattgcccaggcttgcttaggtaaacaaagcagccgggaagctcgaactgggtggagcccaccacagctcaaggaggcctacctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcagggcacactgacacctcacacggcagggtattccaacagacctgcagctgagggtcctgtctgttagaaggaaaactaacaaccagaaaggacatctacacgaaaacccatctgtacatcaccatcatcaaagaccaaaagtagataaaaccacaaagatggggaaaaaacagaacagaaaaactggaaactctaaaacgcagaggccctctcctcctccaaaggaacgcagttcctcaccagcaacagaacaaagctggatggagaatgattttgacgagctgagagaagaaggcttcagacgatcaaattactctgagctacaggaggacattcaaaccaaaggcaaagaagttgaaaactttgaaaaaaatttagaagaatgtataactagaataacc

SEQ ID NO: 10 - DNA, 380 bp, Human
gaaatacagagaacgcgacaaagatactcctcgagaagagcaactcccaagacacataattgtcagattcaccaaagttgaaatgaaggaaaaaatgttaaggcagccagagagaaaggtcgggttaccctcaaagggaagcccatcagactaacagcggctctctcggcagaaaccctacaagccagaagagactgggagccaatattcaacattcttaaaggaaagaattttcaaccccagaatttcatatccagccaaactaagcttcataagtgaaggagaaataaaatactttagagacaagcaaatgctgagagattttgtcaccaccaggcctgccgtaaaagagctcctgaaggaagcgctaaacatggaaa

SEQ ID NO: 11 - DNA, 327 bp, Human
  misc_feature (1)..(327): N = any nucleotide
ggccgggcgcggtggctcacgcctgtaatcccagcactttgggaggccgaggcgggcggatcacggggtcaagagatcgagaccatcccggctaaaacggtgaaaccccgtctctactaaaaatacaaaaaaattagccattgtgtatgnggcgggggcctgtagtccctgggtacttggggggcncagccagnagaatggcgtgatttttgggaggcgtnnnttgnagtgagntttgatccnnttagctgcactcgtgnctgggtgacnnagnnntgctccgtcccnaaaaanaaaacaaaagaaacaaaaaaaaaaaaaanaaaa

(19) United States
(12) Patent Application Publication
(10) Pub. No.: US 2006/0019270 A1
(43) Pub. Date: Jan. 26, 2006

(54) Global DNA methylation assessment using bisulfite PCR

(75) Inventors: Allen S. Yang, Valenica, CA (US); Jean-Pierre Issa, Bellaire, TX (US); Marcos Estecio, Houston, TX (US)

Correspondence Address: David L. Parker;Fulbright & Jaworski L.L.P., Suite 2400, 600 Congress Avenue, Austin, TX, 78701, US

(73) Assignee: Board of Regents The University of Texas System

(21) Appl. No.: 11/096,453

(22) Filed: Apr. 1, 2005

Related U.S. Application Data
(60) Provisional application No. 60/558,742, filed on Apr. 1, 2004.

(51) Int. Cl. C12Q 1/68; C12P 19/34

(52) U.S. Cl. 435/6; 435/91.2

(57) ABSTRACT

The present invention provides a method and assay for determining global methylation using the methylation status of repetitive DNA elements as a surrogate marker. Also provided by the invention is a method for determining efficacy of a DNA methylation inhibiting drug by using changes in repetitive DNA methylation as a marker for drug efficacy. Additionally, the invention provides nucleic acid compositions for performing the method.

### STATEMENT REGARDING FEDERALLY SPONSORED RESEARCH OR DEVELOPMENT

The present invention claims priority to U.S. Provisional Application No. 60/558,742, filed Apr. 1, 2004, which is hereby incorporated by reference in its entirety.

### STATEMENT REGARDING FEDERALLY SPONSORED RESEARCH OR DEVELOPMENT

The present invention was developed with funds from the United States Government, grant numbers P50CA100632 (SPORE), R33CA89837, and CA16672. Therefore, the United States Government may have certain rights in the invention.

### TECHNICAL FIELD

The present invention is related to the fields of molecular biology and genetics. The invention specifically is related to the field of epigenetics. The present invention relates to a method for determining global methylation of DNA.

### BACKGROUND OF THE INVENTION

The conversion of cytosine to 5-methylcytosine is an important epigenetic change in the vertebrate genome (Bird et. al, 1992). DNA methyltransferase can transfer a methyl group from S-adenosyl-methionine to cytosine in CpG dinucleotides. This methylation of cytosine is associated with gene silencing, and genes with abundant 5-methylcytosine in their promoter region are usually transcriptionally silent (Jones et. al, 2001). DNA methylation is vital during development, and aberrant DNA methylation, both hypermethylation and hypomethylation, has been associated with aging, cancer and other diseases (Jones et. al, 2002; Issa et. al, 2002; Richardson et. al, 2003). In addition DNA methylation inhibitors such as 5-azacytidine and decitabine can be used to treat cancer (Glover et. al, 1987; Santini et. al, 2001). Therefore methods to study DNA methylation are important tools in biological research.

There are multiple methods to study DNA methylation. Most of these methods take advantage of a chemical reaction using sodium bisulfite, which can selectively deaminate cytosine but not 5-methylcytosine to uracil (Clark et. al, 1994). This leads to a primary sequence change in the DNA that will allow distinguishing cytosine from 5-methylcytosine. Once this conversion has taken place the sequence differences between a methylated and unmethylated cytosine can be exploited by either direct sequencing, restriction digestion (COBRA)(Xiong et. al, 1997), nucleotide extension assays (MS-SnuPE) (Gonzalgo et. al, 1997), primer specific PCR (MSP) (Herman et. al, 1996), or pyrosequencing (Uhlmann et. al, 2002). These methods are valuable in that they are not labor intensive and require smaller amounts of DNA. However these methods are usually limited in that they can only study a single gene or locus at a time. Earlier methods of using methylation sensitive restriction enzymes and southern blotting to determine gene-specific DNA methylation have largely been replaced by these more convenient methods.

Gene-specific DNA methylation analysis does not provide a global picture of DNA methylation changes within a genome. However, there are several methods of detecting total 5-methylcytosine content in the genome. DNA can be digested into single nucleotides and total genomic 5-methylcytosine can be quantitated by either high-performance liquid chromatography (Wagner et. al, 1981; Feinberg et. al, 1983), thin-layer chromatography (Bestor et. al, 1984), or liquid chromatography/mass spectroscopy (Friso et. al, 2002). Global methylation patterns can also be quantitated using restriction digestion and nearest-neighbor analysis of DNA (Antequera et. al, 1984). Chloracetaldehyde can be used in a fluorescent assay to detect DNA methylation levels (Oakeley et. al, 1999). SssI DNA methyltransferase, which methylates all CpG sites, can be used in conjunction with tritium labeled S-adenosyl methionine to calculate the amount of unmethylated CpG sites and the level of DNA methylation can be inversely determined (Belinsky et. al, 1996). These methods give a sense of global DNA methylation changes, but have the disadvantage of being labor intensive and/or requiring large amounts of good quality DNA as they are not PCR based.

There are approximately 1.4 million Alu repetitive elements in the human genome (Hwu et. al, 1986; Gu et. al, 2000) and a half million LINE-1 elements (Kazazian et. al, 2002) that are normally heavily methylated, and it is estimated that more than one third of DNA methylation occurs in repetitive elements (Kochanek et. al, 2002; Schmid et. al, 1998; Bestor et. al, 1998).

5-aza-2′-deoxycytidine (decitabine) is a pyrimidine analog first synthesized almost 40 years ago. Early clinical trials showed that decitabine had consistent clinical activity in patients with myeloid leukemia. There is considerable experience in the use of decitabine and a similar drug 5-azacytidine in numerous clinical trials in patients with CML, AML and Myelodysplastic Syndrome (Glover, Leyland-Jones et al. 1987; Santini, Kantarjian et al. 2001). More importantly 5-azacytidine was shown to prolong survival with an improved quality of life in patients with MDS in a randomized controlled trial (Silverman, Demakos et al. 2002). Recent efforts have been focused on giving lower doses of decitabine to minimize toxicity and to take advantage of the unique property of azacytidine and deictabine to inhibit DNA methylation (Wijermans, Lubbert et al. 2000; Issa, Garcia-Manero et al. 2004).

### BRIEF SUMMARY OF THE INVENTION

An embodiment of the invention is a method of determining the methylation status of one or more repetitive DNA elements of a DNA molecule comprising: obtaining the DNA molecule; and analyzing the methylation status of one or more of the modified repetitive DNA elements. In a specific embodiment, the analyzing step comprises reacting the DNA molecule with a modifying agent to convert unmethylated cytosine residues to uracil residues;

In a specific embodiment, the analyzing step further comprises amplifying one or more of the modified repetitive DNA elements. In a specific embodiment, the method further comprises the step of digesting the amplified one or more repetitive DNA elements with a restriction enzyme. In a further specific embodiment, the amplifying step comprises using a forward consensus primer and a reverse consensus primer to the modified DNA molecule. In one embodiment of the invention, the forward consensus primer and the reverse consensus primer comprise a restriction site at the 5-prime end. In a specific embodiment, the restriction site is the MboI restriction site. In another specific embodiment, the forward consensus primer comprises a linker sequence at the 5-prime end.

In an embodiment of the invention, the forward consensus primer comprises SEQ ID NO: 1 and the reverse consensus primer comprises SEQ ID NO:2. In another embodiment of the invention, the forward consensus primer comprises SEQ ID NO:3 and the reverse consensus primer comprises SEQ ID NO:4. In another embodiment of the invention, the forward consensus primer comprises SEQ ID NO:7 and the reverse consensus primer comprises SEQ ID NO:8.

In one embodiment of the invention, the methylation status of at least about 1,000 repetitive DNA elements is analyzed. In another embodiment of the invention, the methylation status of at least about 10,000 repetitive DNA elements is analyzed. In specific embodiments of the invention, the repetitive DNA elements may be SINE elements, LINE elements, or Alu elements.

In an embodiment of the invention, the modifying agent comprises sodium bisulfite.

In an embodiment if the invention, the analyzing of the modified DNA molecule comprises Southern blotting, restriction digest analysis, mass spectrometry, or a sequencing reaction.

An embodiment of the invention is a method of monitoring efficacy of a methylation inhibiting drug in a patient comprising: obtaining a DNA molecule with one or more repetitive DNA elements from a patient treated with a methylation inhibiting drug; analyzing the methylation status of one or more of the repetitive DNA elements; and comparing the methylation status of the one or more repetitive DNA elements to a control. The control is a DNA molecule taken from the patient before treatment with the methylation inhibiting drug in one embodiment of the invention.

In an embodiment of the invention, the methylation inhibiting drug is decitabine, 5-azacytidine, hydralazine or procainamide.

An embodiment of the invention is a method of diagnosing a disease associated with a change in methylation status comprising: obtaining a DNA molecule with one or more repetitive DNA elements from a patient; analyzing the methylation status of one or more of the repetitive DNA elements; and comparing the methylation status of the one or more repetitive DNA elements to a control.

An embodiment of the invention is a primer comprising a consensus sequence of a modified DNA repetitive element. Other embodiments of the invention are nucleic acid compositions comprising SEQ ID NO:1 and SEQ ID NO:2; SEQ ID NO:3 and SEQ ID NO:4; or SEQ ID NO:7 and SEQ ID NO:8.

An embodiment of the invention is a kit for determining global methylation of a genomic DNA sample comprising at least one primer comprising a consensus sequence of a modified DNA repetitive element

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1A-FIG. 1B show direct DNA sequencing of Bisulfite Repetitive Element PCR of Alu elements. FIG. 1A shows a schematic of possible fates of Alu element CpG methylation sites. Due to the mutation of CpG sites via spontaneous deamination of 5-methylcytosine to T during evolution a CpG site can be changed into a TpG or CpA dinucleotide. Neither TpG nor CpA are targets for methylation. Following bisulfite treatment a methylated CpG will remain CpG, however an umethylated CpG will give rise to TpG, which is indistinguishable from a deamination mutation of the forward strand. Thus three possiblites arise for the sequencing data: CpG that represents a methylated CpG site, TpG which represents either an unmethylated CpG site or a mutation of the forward strand, and TpA which represent a mutation of the reverse strand followed by conversion of the unmethylated C to T by bisulfite. FIG. 1B shows sequencing data of Bisulfite Repetitive Element PCR of Alu elements. Genomic DNA was isolated from peripheral human blood and bisulfite treated. Alu element PCR was performed and the PCR product was cloned and 15 clones were sequenced. There were 12 potential CpG methylation sites per clone for a total of 180 potential methylation sites sequenced. Black circles represent methylated CpG sites (66/180=36.7%). “T” represents TpG sites that were either unmethylated or mutated (53/180=24.4%). “A” represents TpA sites that were mutated (41/180=22.8%). “X” represents other mutations (20/180=11.1%). The CpG sites used for pyrosequencing (“PS”) and COBRA (“MboI”) are indicated;

FIG. 2 is a calculation of the number of Alu elements being assessed by Bisulfite Alu PCR. Competitive PCR was performed using a fixed amount of bisulfite treated genomic DNA and a plasmid containing a cloned Alu element fragment with an internal duplication that gives a larger PCR product. A fixed amount of bisulfite treated genomic DNA was mixed with serial dilutions of the plasmid which gave two PCR products, one from the genomic DNA and a larger one from the plasmid. The PCR products were quantitated using a capillary electrophoresis system, the Agilent 2100 Bioanalyzer (Agilent Technologies, Palo Alto, Calif.) and plotted below. From this experiment it is approximated that 0.05 ng of plasmid gives equivalent PCR product to 50 ng of bisulfite treated genomic DNA;

FIG. 3 illustrates the quantitation of DNA methylation in cell lines treated with 5-aza-2′deoxcytidine (DAC) using the Bisulfite Repetitve Element PCR technique. Hct116, RKO and SW48 cell lines were treated with 5-aza-2′deoxcytidine (DAC). Genomic DNA was isolated from DAC treated (+) and untreated (−) controls. The genomic DNA was treated with sodium bisulfite and a non-specific PCR was performed which amplified a pool of Alu or LINE-1 repetitive elements. The PCR product was then digested with MboI (Alu) or HinfI (LINE-1), which only cuts repetitive elements that were originally methylated. The Alu PCR assays a single methylation site and therefore the MboI digestion will cut the PCR product from 152 to 125 and 27 basepairs. The LINE-1 PCR assays the methylation of two sites and therefore HinfI can generate five possible digestion products of 285, 247, 166, 128 and 38 bp (not seen). The digested PCR product was separated by polyacrylamide gel electrophoresis and stained with ethidium bromide. The lower cut bands represent methylated repetitive elements. The upper band represents unmethylated repetitive elements or repetitive elements in which the restriction site has been mutated. The PCR bands were quantitated and the amount of methylation is shown below each gel lane. Similar results were obtained for both the Alu element and LINE-1 assays;

FIG. 4A-FIG. 4C show quantitation of DNA methylation using Bisulfite Repetitive Element PCR and pyrosequencing. SssI methylase treated DNA (FIG. 4A), untreated Hct116 cell line DNA (FIG. 4B), or DAC treated Hct116 cell line DNA (FIG. 4C) was bisulfite treated and PCR of Alu repetitve elements was performed. The PCR product was purified and methylation was quantitated using the PSQ HS 96 Pyrosequencing System (Pyrosequencing, Inc.; Westborough, Mass.). The pyrogram quantitates C for methylated and T for unmethylated or mutated DNA. The shaded regions represent three tandem CpG sites quantitated in Alu elements, and the percent methylation at each site is shown above the peaks. The average methylation of the three sites is calculated on the left for each pyrogram. The maximum absolute methylation of 23.2% is calculated by SssI treated DNA (FIG. 4A), Hct116 cells have 20.2% methylation, and this methylation decreases to 14.5% after DAC treatment of Hct116 cells.;

FIG. 5A-FIG. 5B show COBRA of Alu repetitive elements. FIG. 5A shows COBRA of Alu Repetitive elements performed on normal colon mucosa from patients of various ages. An age dependent decrease in Alu element methylation was observed. This age dependent decrease in Alu element methylation was statistically significant with a Multiple R=0.59 and Significance F=0.00007. FIG. 5B shows COBRA of Alu Repetitive elements performed on matched sets of normal colon mucosa and colon cancers from patients of various ages. There is not an age dependent decrease in Alu element methylation in colon cancers (Multiple R=0.28, Significance F=0.13);

FIG. 6 illustrates treatment schema of patient samples. Patients were treated as part of a Phase II study of Decitabine which was given initially a dose of 90 mg/m2 twice daily for 5 consecutive days. This dose was later decreased to 50 mg/m2 twice daily due to toxicity. In this “High” Dose Study a total dose of 500-900 mg/m2 of decitabine was given over a 5 day period. Peripheral blood specimens were collected on day 0 or 1 prior to treatment, days 2-4 during treatment, and on days 5 or 6 after the completion of treatment. In the second study, a Phase I strategy of “Low” Dose Decitabine was used. This study was designed to take advantage of Decitabine's demethylating properties. Escalating doses of Decitabine of 5, 10, 15 and 20 mg/m2 were given as ten daily doses over a two week period. The total dose delivered was only 50 to 200 mg/m2 over a 14 day period. Peripheral blood specimens were collected on day 0 or 1 prior to treatment, and during days 2-14 of treatment;

FIG. 7 is an example of methylation analysis of leukemia patients treated with Decitabine Methylation of peripheral blood from leukemia patients was assessed for sequential days of decitabine treatment. Methylation was quantitated by bisulfite-PCR followed by restriction enzyme digestion. FIG. 7 shows an analysis of Alu elements was quantitated using a capillary electrophoresis system. The arrows indicate methylated bands/peaks;

FIG. 8A-FIG. 8B depict DNA methylation changes induced by Decitabine. DNA methylation was quantitated using COBRA of Alu Repetitive Elements and three gene promoter regions for both the High Dose Study (FIG. 8A) and the Low Dose Study (FIG. 8B). A non specific COBRA of Alu repetitive elements which are known to be heavily methylated allowed us to assess the methylation of several thousand loci simultaneously. Gene specific COBRA of three different types of genes were analyzed. A gene locus which is heavily methylated (HOX A5), an imprinted gene (H19), and a gene which is normally unmethylated but becomes aberrantly methylated in leukemia (p15). Although demethylation was observed in some patients for all the loci examined, consistent results were only obtained using the Alu Element Assay;

FIG. 9A-FIG. 9B show demethylation dose response of Decitabine. FIG. 9A illustrates that demethylation of Alu elements was observed in both the High and Low Dose Decitabine studies. There was more demethylation observed in the High Dose Study and Demethylation seemed to plateau after 5 to 8 days. FIG. 9B shows DNA methylation at days 5 to 6 for both studies was compared to the dose given. Approximately 15 to 20 mg/m2/day appeared to be the optimal demethylating dose with no significant decrease in methylation with higher doses of Decitabine. Note only one patient sample was available of the patients treated at 20 mg/m2/day and 180 mg/m2/day.

FIG. 10A-FIG. 10B show demethylation and response. Demethylation of Alu elements was compared in those patients who responded to Decitabine and those who did not respond to Decitabine for both the High Dose Study (FIG. 10A) and the Low Dose Study (FIG. 10B). In the High Dose Study demethylation did not correlate to response, and surprisingly there was a trend for non-responders to have a greater decrease in methylation. (p=0.23). In the Low Dose Study demethylation did correlate to response with patients who had more demethylation responding to Decitabine therapy. (p=0.04 days 5-8 and p=0.02 days 9-14).

FIG. 11A-FIG. 11B show methylation status of Alu and LINE-1 elements in cell lines and patients. FIG. 11A shows Alu and LINE-1 Methylation in Primary Tumors and Cancer Cell Lines. FIG. 11B shows demethylation of Alu elements by 5-aza-2′deoxycytidine in patients with leukemia

FIG. 12A-FIG. 12B illustrate the LINE-1 sequence before and after bisulfite treatment. In FIG. 12A, the target amplified sequence is indicated and in italicized letters are the HinfI enzyme cutting sites. FIG. 12B is the graphic view of the LINE-1 methylation assay.

FIG. 13 shows methylation status of LINE-1 promotor in peripheral blood lymphocytes (PBL) and matched tumor and normal colon tissue. PCR products were cloned and five independent clones were sequenced per case.

FIG. 14 shows quantitation of LINE-1 DNA methylation in cell lines treated with 5-aza-2′deoxcytidine (DAC) using the LINE-1 methylation assay. Hct116, RKO and SW48 cell lines were treated with 5-aza-2′deoxycytidine (DAC). The genomic DNA was treated with sodium bisulfite and a non-specific PCR was performed which amplified a pool of LINE-1 repetitive elements. The PCR product was then digested with HinfI, which only cuts repetitive elements that were originally methylated. The LINE-1 PCR assays the methylation of two sites and therefore HinfI can generate five possible digestion products of 285, 247, 166, 128 and 38 bp (not seen). The digested PCR product was separated by polyacrylamide gel electrophoresis and stained with ethidium bromide. The PCR bands were quantitated and the amount of methylation is shown below each gel lane.

FIG. 15 is a graphic representation of methylation density in PBL and cancer cell lines from different tissues and matched cancer and normal tissue from colon cancer patients. The methylation degree was lower in cancer tissue compared to normal tissue in all cases.

FIG. 16A-FIG. 16D depict graphic representations of LINE-1 methylation in normal adjacent tissue and tumor colon samples (FIG. 15A) and in different tumor stages (FIG. 16B). Normal adjacent mucosa of tumor presenting MLH1 methylation and consequent MSI+ phenotype are more demethylated according LINE-1 retrotranposons methylation (FIG. 16C). In the CIMP+/MSI+ group, there was no difference in LINE-1 methylation between normal adjacent and cancer tissues, while CIMP+/MSI− and CIMP− cases presented decrease in LINE-1 methylation between normal adjacent and cancer tissues. This result could be partially due to the fact that LINE-1 methylation was lower in the normal adjacent tissue of the CIMP+/MSI+ group (FIG. 16D).

### DETAILED DESCRIPTION OF THE INVENTION

I. Definitions

As used herein, the use of the word “a” or “an” when used in conjunction with the term “comprising” in the sentences and/or the specification may mean “one,” but it is also consistent with the meaning of “one or more,” “at least one,” and “one or more than one.” As used herein “another” may mean at least a second or more. Still further, the terms “having”, “including”, “containing” and “comprising” are interchangeable and one of skill in the art is cognizant that these terms are open ended terms.

A “methylation-inhibiting drug” is a drug that reactivates expression of epigenetically silenced regions of DNA can be a methyltransferase inhibitor (e.g., 5-aza-2′-deoxycytidine; DAC, procainamide), a histone deacetylase inhibitor (e.g., trichostatin A; TSA), or a combination of drugs such as a combination of DAC and TSA. Methylation inhibiting drugs contemplated in the present incvention include decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA

The term “modifies” as used herein refers to the conversion of an unmethylated cytosine to another nucleotide which will distinguish the unmethylated from the methylated cytosine.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule.

The term “primer” as used herein refers to a sequence comprising two or more deoxyribonucleotides or ribonucleotides, preferably more than three, and most preferably more than 8, which sequence is capable of initiating synthesis of a primer extension product, which is substantially complementary to a designated nucleic acid, or, in some embodiments of the invention, a plurality of designated nucleic acids. In one embodiment of the invention, the designated nucleic acid is a SINE element or LINE element. In a specific embodiment, the designated nucleic acid is an Alu element.

“Repetitive genomic DNA elements” or “repetitive DNA elements” are families of related sequences that occur in up to thousands of copies in the genome. Due to their abundance they are represented in virtually every piece of genomic DNA from higher eukaryotic organisms. Repetitive DNA elements contemplated by the present invention included SINEs and LINEs. A “plurality of repetitive DNA elements” as used herein refers to more than one repetitive DNA element. In a particular embodiment of the invention, a plurality of repetitive DNA elements is amplified using a forward and reverse primer. The amplified nucleic acids comprise a pool of nucleic acids, each potentially having polymorphisms or differentially methylated CpG dinucleotides. Amplifying a plurality of repetitive DNA elements, as described herein, encompasses amplification of a segment of repetitive DNA element, as well as the full-length element.

II. Detailed Embodiments of the Invention

The present invention relates to an assay that provides methylation status of repetitive DNA elements as a surrogate marker for global DNA methylation. The invention also provides methylation status of a plurality of repetitive DNA elements in an individual being treated with a methylation-inhibiting drug. The invention provides nucleic acid compositions useful for determining methylation status of repetitive DNA elements. It is contemplated that determination of methylation status of repetitive DNA elements as a surrogate marker for global DNA methylation and can serve to monitor, predict, or diagnose a number of clinical diseases or biological states including but not limited to cancer, predisposition to cancer, genetic or inherited disorders, diseases associated with metabolic disorders, diseases associated with nutritional deficiency/status, or diseases associated with aging.

One with skill in the art realizes that the invention can provide methylation status of a variety of different repetitive DNA elements, such as SINE and LINE elements. SINE elements are Short Interspersed Nuclear Elements (SINEs). An example of a SINE element is an Alu element. Alu elements are characterized by length of approximately 280 bp and are generally GC-rich. They are often located in untranslated intronic regions in the DNA. Long Interspersed Nuclear Elements (LINEs) are generally characterizes as AT rich regions and are 6-8 kilobases in length. LINEs contain internal promotors for RNA polymerase III. According to the present invention, any repetitive DNA element is considered appropriate as a surrogate marker for global DNA methylation. The invention also may provide methylation status for multiple copy genes, mitochondrial DNA sequences, or any sequence in which there are multiple copies in the genome. Examples of repetitive DNA elements contemplated by the present invention are SEQ ID NO:9, SEQ ID NO: 10, and SEQ ID NO: 11.

The invention provides primers which correspond to consensus sequences of repetitive DNA elements. Consensus sequences in repetitive DNA elements are sequences that are substantially similar from element to element. In certain embodiments of the invention, a consensus sequence is at least 4, at least 6, at least 8, at least 10, or at least 12 nucleotides in length. One with skill in the art realizes that the consensus sequence may be at least 30%, 40%, 50%, 60%, 70%, 80%, 90%, or 100% conserved between the repetitive DNA elements. In an embodiment of the invention, the repetitive DNA element comprises at least a first and a second consensus sequence, wherein the second sequence is downstream of the first sequence.

In an embodiment of the invention, a forward consensus primer and a reverse consensus primer are designed to correspond to the first and the second consensus sequences and to amplify the nucleic acid region between them. The forward and reverse consensus primers thus amplify the nucleic acid regions between the two consensus sequences anywhere they appear in the genome. As consensus sequences in repetitive DNA elements are found at multiple locations in the genome, primers designed to correspond to such sequences are capable of amplifying a plurality of repetitive DNA regions in the genome. For example, the primer pair SEQ ID NO: 1 and SEQ ID NO:2 amplifies approximately 15,000 Alu elements.

As a consensus sequence may comprise double-stranded DNA, the forward primer is complementary to the bottom strand, and the reverse primer is the reverse complement of the top strand, as is well known to one with skill in the art. Therefore, a single primer pair is able to amplify a plurality of nucleic acid regions from repetitive DNA elements, including polymorphic regions. In a preferred embodiment of the invention, the consensus sequence is designed after taking into account DNA modification by a modifying agent, wherein any unmethylated cytosines are converted to uracil. Thus, a repetitive DNA element having the unmodified sequence CCCCCCC would be converted to UUUUUUU with a modifying agent, thus causing the design of a consensus forward primer to be TTTTTTT. Similarly, to design a reverse primer to modified DNA starting with the unmodified sequence CCCCCCC, the primer would have the reverse complementary sequence AAAAAAA. Therefore, unmodified starting sequences CCCCCCC and TTTTTTT would have the same forward consensus primer after DNA modification. In an embodiment of the invention, the consensus primers are designed to minimize the number of potential CpG dinucelotides in the starting, or “unmodified” sequence in order to eliminate amplification bias towards methylated or unmethylated regions. Consensus primers may also comprise linking regions, flanking regions, or restriction digest sequences. Examples of consensus primers according to the present invention are SEQ ID NO: 1 and SEQ ID NO:2, SEQ ID NO:3 and SEQ ID NO:4, and SEQ ID NO:7 and SEQ ID NO:8.

In a preferred embodiment of the invention, a DNA molecule is modified such that unmethylated cytosines are coverted to uracil. Preferably, the agent used for modifying umethylated cytosine is sodium bisulfite, however, other agents that similarly modify unmethylated cytosine, but not methylated cytosine can also be used in the method of the invention. Sodium bisulfite (NaHSO₃) reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by Taq polymerase and therefore upon PCR, the resultant product contains cytosine only at the position where 5-methylcytosine occurs in the starting template DNA.

III. Methods of Methylation Analysis

In certain embodiments of the invention, analysis of methylation status of one or more repetitive DNA elements is contemplated. Methylation states at one or more CpG methylation sites within a DNA sequence include “unmethylated,” “fully-methylated,” “hypomethylated,” “hypermethylated,” “decreased methylation,” “increased methlyation,” and “hemi-methylated.”

The repetitive DNA element may be hypermethylated, which refers to the methylation state corresponding to an increased presence of 5-methylcytosine at one or a plurality of CpG dinucleotides within a DNA sequence of a test DNA sample, relative to the amount of 5-methylcytosine found at corresponding CpG dinucleotides within a normal control DNA sample.

The repetitive DNA element may be hypomethylated, which refers to the methylation state corresponding to a decreased presence of 5-methylcytosine at one or a plurality of CpG dinucleotides within a DNA sequence of a test DNA sample, relative to the amount of 5-methylcytosine found at corresponding CpG dinucleotides within a normal control DNA sample.

Below are outlined a number of techniques known to one with skill in the art for determining methylation status of a given DNA molecule.

(1) MS-PCR Technique

In the PCR technique, specific primers are designed and synthesized so that a particular region in genomic DNA is amplified. Using these primers, a PCR is performed with the particular region as a template. When the regions of the genomic DNA have been digested with a methylation-sensitive restriction enzyme before the amplification, methylated genes are not cut while unmethylated genes are cut. These genes are amplified by PCR, and the amplified fragments are separated by electrophoresis. Then, the resultant bands are examined. If the test gene is methylated, bands are observed. If the test gene is unmethylated, no bands are observed. Using this fact, whether the test gene is methylated or not can be ascertained.

(2) Southern Blotting

Southern blot analysis is a method by which the presence of DNA sequences in a restriction endonuclease digest of DNA or DNA-containing composition is confirmed by hybridization to a known, labeled oligonucleotide or DNA fragment. Southern analysis typically comprises electrophoretic separation of DNA digests on agarose gels, denaturation of the DNA after electrophoretic separation, and transfer of the DNA to nitrocellulose, nylon, or another suitable membrane supports for analysis with a radiolabeled, biotinylated or enzyme-labeled probe as described in sections 9.37-9.52 of Sambrook et al, supra.

When genomic DNA is digested with a methylation-sensitive restriction enzyme, methylated restriction sites are not cut while unmethylated restriction sites are cut. The digested genomic DNA is separated by agarose electrophoresis. The DNA fragments are transferred onto a nylon membrane followed by hybridization with a ³²P-labeled gene-specific probe. Then, the presence or absence of methylation in the gene used as the probe can be detected using the difference in length of the detected bands. Examples of methylation sensitive restriction endonucleases which can be used to detect 5′CpG methylation include SmaI, SacII, EagI, MspI, HpaII, BstUI and BssHII, for example

(3) CpG Island Array Technique

First, genomic DNA is digested with a restriction enzyme which does not contain methylatable sequences in its recognition site. Then, a linker containing a primer site for PCR is ligated to the digested genomic DNA. The linker-ligated DNA fragments are digested with a methylation-sensitive restriction enzyme and then amplified by PCR utilizing the primer site in the linker. At that time, unmethylated genes are cut between primers by the methylation-sensitive restriction enzyme, and not amplified by PCR. On the other hand, only methylated genes are amplified. Thus, if such a PCR reaction is performed using a combination of any two tissues or cells, the types of amplified genes are different because of the existence of methylated regions specific to respective tissues or cells. Only those genes that exhibit difference in methylation between the tissues or cells are selected by the subtraction method and used as probes. These probes are hybridized with a gene library to thereby confirm their nucleotide sequences. Thus, the genes can be identified.

(4) Pyrosequencing

Pyrosequencing™ is a real-time, sequencing by synthesis method catalyzed by four kinetically well-balanced enzymes, DNA polymerase, ATP sulfurylase, luciferase, and apyrase. It differs from Sanger's sequencing method in the order of nucleotide incorporation. Each nucleotide is dispensed and tested individually for its incorporation into a nascent DNA template. Each incorporation event is accompanied by release of pyrophosphate (PPi) in a quantity equimolar to the amount of nucleotide incorporated. ATP sulfurylase quantitatively converts PPi to ATP in the presence of adenosine 5′ phosphosulfate. ATP then drives the luciferase-mediated conversion of luciferin to oxyluciferin that generates visible light in amounts that are proportional to the amount of ATP. The light is detected by a charge coupled device (CCD) camera and displayed as a peak in a pyrogram™. Each peak height is proportional to the number of nucleotides incorporated. Unincorporated dNTP and excess ATP are continuously degraded by Apyrase. After the degradation is completed, the next dNTP is added and a new Pyrosequencing™ cycle is started. As the process continues, the complementary DNA strand is built up. To pyrosequence an unknown DNA sequence, a cyclic nucleotide dispensation order (NDO) is generally used. As a result of each cycle of dATP, dGTP, dCTP and dTTP dispensation, one of the four dNTPs is incorporated into the DNA template while the other dNTPs are degraded by Apyrase. When a DNA sequence is known, non-cyclic NDOs can be programmed with predictable pyrograms. Nucleotide sequence is determined from the order of nucleotide dispensation and peak height in the pyrogram.

In the case of CpG methylation analysis, Pyrosequencing™ can be used to determine a percentage of cytosine incorporation compared to thymine incorporation at a given potential methylation site in a pool of sequences, such as a plurality of Repetitive DNA elements amplified from sodium bisulfite treated DNA by consensus primers.

IV. Purification of Nucleic Acids

A nucleic acid may be purified on polyacrylamide gels, cesium chloride centrifugation gradients, or by any other means known to one of ordinary skill in the art (see for example, Sambrook et al., 1989, incorporated herein by reference).

In certain aspect, the present invention concerns a nucleic acid that is an isolated nucleic acid. As used herein, the term “isolated nucleic acid” refers to a nucleic acid molecule (e.g., an RNA or DNA molecule) that has been isolated free of, or is otherwise free of, the bulk of the total genomic and transcribed nucleic acids of one or more cells. In certain embodiments, “isolated nucleic acid” refers to a nucleic acid that has been isolated free of, or is otherwise free of, bulk of cellular components or in vitro reaction components such as for example, macromolecules such as lipids or proteins, small biological molecules, and the like.

The nucleic acid-containing specimen, such as a DNA molecule, used for detection of methylated CpG may be from any source including brain, colon, urogenital, hematopoietic, thymus, testis, ovarian, uterine, prostate, breast, colon, lung and renal tissue and may be extracted by a variety of techniques such as that described by Maniatis, et al (Molecular Cloning: A Laboratory Manual, Cold Spring Harbor, N.Y., pp 280, 281, 1982). If the extracted sample is impure (such as plasma, serum, or blood or a sample embedded in parrafin), it may be treated before amplification with an amount of a reagent effective to open the cells, fluids, tissues, or animal cell membranes of the sample, and to expose and/or separate the strand(s) of the nucleic acid(s). This lysing and nucleic acid denaturing step to expose and separate the strands will allow amplification to occur much more readily.

One with skill in the art also realizes that methylation of repetitive DNA elements occurs in a variety of species, such as mice, and DNA molecules from species in which repetitive DNA elements are normally methylated are contemplated for use in the present invention.

V. Nucleic Acid Segments

In certain embodiments, nucleic acids described in the present invention are nucleic acid segments. Nucleic acid segments are smaller fragments of a nucleic acid, such as for non-limiting example, those that encompass part of an Alu element or LINE-1 element. In certain embodiments of the invention, it is contemplated that nucleic acid segments of repetitive DNA elements will be amplified in order to determine the methylation status of the fragments. For example, a forward consenus primer and a reverse consensus primer will amplify a region of DNA that is approximately 100 bp, 150 bp, 200 bp, or 250 bp. Such nucleic acid segments that are not full-length versions of repetitive DNA elements are sufficient to provide methylation status of the full-length element.

Various nucleic acid segments may be designed based on a particular nucleic acid sequence, and may be of any length. By assigning numeric values to a sequence, for example, the first residue is 1, the second residue is 2, etc., an algorithm defining all nucleic acid segments can be created: [n to n+y] where n is an integer from 1 to the last number of the sequence and y is the length of the nucleic acid segment minus one, where n+y does not exceed the last number of the sequence. Thus, for a 10-mer, the nucleic acid segments correspond to bases 1 to 10, 2 to 11, 3 to 12 . . . and so on. For a 15-mer, the nucleic acid segments correspond to bases 1 to 15, 2 to 16, 3 to 17 . . . and so on. For a 20-mer, the nucleic segments correspond to bases 1 to 20, 2 to 21, 3 to 22 . . . and so on. In certain embodiments, the nucleic acid segment may be a probe or primer. As used herein, a “probe” generally refers to a nucleic acid used in a detection method or composition. As used herein, a “primer” generally refers to a nucleic acid used in an extension or amplification method or composition.

VI. Nucleic Acid Complements

The present invention also encompasses a nucleic acid that is complementary to a nucleic acid. In one embodiment, the invention encompasses a nucleic acid or a nucleic acid segment complementary to a nucleic acid segment of an Alu element, a LINe-1 element, or other repetitive DNA element. A nucleic acid is “complement(s)” or is “complementary” to another nucleic acid when it is capable of base-pairing with another nucleic acid according to the standard Watson-Crick, Hoogsteen or reverse Hoogsteen binding complementarity rules.

VII. Primers

The primers of the invention embrace oligonucleotides of sufficient length and appropriate sequence so as to provide specific initiation of polymerization on a significant number of nucleic acids in the polymorphic locus. Environmental conditions conducive to synthesis include the presence of nucleoside triphosphates and an agent for polymerization, such as DNA polymerase, and a suitable temperature and pH. The primer is preferably single stranded for maximum efficiency in amplification, but may be double stranded. If double stranded, the primer is first treated to separate its strands before being used to prepare extension products. Preferably, the primer is an oligodeoxy ribonucleotide. The primer must be sufficiently long to prime the synthesis of extension products in the presence of the inducing agent for polymerization. The exact length of primer will depend on many factors, including temperature, buffer, and nucleotide composition. The oligonucleotide primer typically contains 12-20 or more nucleotides, although it may contain fewer nucleotides.

Primers of the invention are designed to be “substantially” complementary to each strand of the genomic locus to be amplified and include the appropriate G or C nucleotides as discussed above. This means that the primers must be sufficiently complementary to hybridize with their respective strands under conditions which allow the agent for polymerization to perform. In other words, the primers should have sufficient complementarity with the 5′ and 3′ flanking sequences to hybridize therewith and permit amplification of the genomic locus.

Oligonucleotide primers of the invention are employed in the amplification process which is an enzymatic chain reaction that produces exponential quantities of target locus relative to the number of reaction steps involved. Typically, one primer is complementary to the negative (−) strand of the locus and the other is complementary to the positive (+) strand. Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

The oligonucleotide primers of the invention may be prepared using any suitable method, such as conventional phosphotriester and phosphodiester methods or automated embodiments thereof. In one such automated embodiment, diethylphosphoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066.

In specific embodiments of the invention, the primers described herein are complementary to repetitive DNA elements, such as Alu elements or LINE elements. The primers contemplated may comprise additional functional sequences, such as restriction sites, flanking sequences, or linking sequences. In a specific embodiment, the primers comprise a restriction site at the 5-prime end.

VIII. Methods of Amplification

Where the target nucleic acid sequence of the sample contains two strands, it is necessary to separate the strands of the nucleic acid before it can be used as the template. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80° C. to 105° C. for times ranging from about 1 to 10 minutes. Strand separation may also be induced by an enzyme from the class of enzymes known as helicases or by the enzyme RecA, which has helicase activity and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

When complementary strands of nucleic acid or acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, preferably at a pH of 7-9, most preferably about 8. Preferably, a molar excess (for genomic nucleic acid, usually about 10⁸:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. A large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90°-100C from about 1 to 10 minutes, preferably from 1 to 4 minutes. After this heating period, the solution is allowed to cool to room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40° C. Most conveniently the reaction occurs at room temperature.

The agent for polymerization may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation). Suitable enzymes will facilitate combination of the nucleotides in the proper manner to form the primer extension products which are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

Preferably, the method of amplifying is by PCR, as described herein and as is commonly used by those of ordinary skill in the art. Alternative methods of amplification have been described and can also be employed as long as the methylated and non-methylated loci amplified by PCR using the primers of the invention is similarly amplified by the alternative means.

The amplified products are preferably identified as methylated or non-methylated by sequencing. Sequences amplified by the methods of the invention can be further evaluated, detected, cloned, sequenced, and the like, either in solution or after binding to a solid support, by any method usually applied to the detection of a specific DNA sequence such as PCR, oligomer restriction (Saiki, et al, Bio/Technology, 3:1008-1012, 1985), allele-specific oligonucleotide (ASO) probe analysis (Conner, et al., Proc. Natl. Acad. Sci. USA, 80:278, 1983), oligonucleotide ligation assays (OLAs) (Landegren, et al., Science, 241:1077, 1988), and the like. Molecular techniques for DNA analysis have been reviewed (Landegren, et al., Science, 242:229-237, 1988).

IX. Kits

Any of the compositions described herein may be comprised in a kit. In a non-limiting example, a nucleic acid composition and/or additional agent, may be comprised in a kit. The kits will thus comprise, in suitable container means, a nucleic acid composition and/or an additional agent of the present invention.

The kits may comprise a suitably aliquoted nucleic acid composition and/or additional agent of the present invention, whether labeled or unlabeled, as may be used to prepare a standard curve for a detection assay. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the nucleic acid composition and/or additional agent and any other reagent containers in close confinement for commercial sale. Such containers may include injection or blow-molded plastic containers into which the desired vials are retained.

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### DNA and Cell Lines

Hct116, RKO and SW48, colon cancer cell lines (American Type Culture Collection, Manassas, Va.), were cultured using standard methods. Cells were treated with 5 μM 5-aza-2′deoxycytidine (DAC) for three days prior to being harvested. DNA from cell lines and peripheral blood leukocytes was extracted using phenol-chloroform extraction methods well known to one with skill on the art.

### Example 2

### Bisulfite Treatment

Bisulfite modification of genomic DNA is known in the art (see Clark et. al, 1994). In brief, 1.5 μg prepared 10 mM hydroquinone (Sigma) and 520 μl of 3M Sodium Bisulfite (Sigma) at pH=5.0 were added and mixed. The samples were overlayed with mineral oil to prevent evaporation and incubated at 50° C. for 16 hours. The bisulfite treated DNA was isolated using Wizard DNA Clean-Up System (Promega). The DNA was eluted by 50 μl of warm water and 5.5 μl of 3M NaOH were added for 5 minutes. The DNA was ethanol precipitated with glycogen as a carrier and resuspended in 20 μl water. Bisulfite treated DNA was stored at −20° C. until ready for use.

### Example 3

### PCR of Repetitive Elements

Methylation analysis of Alu repetitive elements was performed initially by the COBRA assay. A 25 μl PCR reaction was carried out in 60 mM Tris-HCl pH=9.5, 15 mM ammonium sulfate, 5.5 mM MgCl2, 10% DMSO, 1 mM dNTP mix, 1 unit of Taq polymerase, 50 pmol of the forward primer (5′-GATCTTTTTATTAAAAATATAAAAATTAGT-3′) (SEQ ID NO: 1), 50 pmol of the reverse primer (5′-GATCCCAAACTAAAATACAATAA-3′) (SEQ ID NO: 2), and approximately 50 ng of bisulfite treated genomic DNA. The best COBRA results were obtained if the PCR primers contained a restriction site on the 5′ end that would be recognized by the COBRA restriction enzyme, which digested non-specific PCR products and helped prevent primer dimer formation. PCR cycling conditions were 96° C. for 90 seconds, 43° C. for 60 seconds, and 72° C. for 120 seconds for 27 cycles. The PCR product was then digested with 10 U of MboI. The digested PCR product was then separated by polyacrylamide gel electrophoresis or the PCR products were quantitated using a capillary electrophoresis system, an Agilent 2100 Bioanalyzer (Agilent Technologies, Palo Alto, Calif.).

The Alu element PCR reaction was modified for pyrosequencing based methylation analysis. A 50 μl PCR was carried out in 60 mM Tris-HCl pH=8.5, 15 mM Ammonium Sulfate, 2 mM MgCl₂, 10% DMSO, 1 mM dNTP mix, 1 unit of Taq polymerase, 5 pmol of the forward primer (5′-GGGACACCGCTGATCGTATATTTTTATTAAAAATATAAAAATTAGT-3′) (SEQ ID NO: 3), 50 pmol of the reverse primer (5′-CCAAACTAAAATACAATAA-3′) (SEQ ID NO: 4), 50 pmol of biotinylated universal primer (5′-GGGACACCGCTGATCGTATA-3′) (SEQ ID NO: 5), and approximately 50 ng of bisulfite treated genomic DNA. The forward primer has a 20 bp linker sequence on the 5′ end that is recognized by a biotin labeled primer so the final PCR product can be purified using sepharose beads. The PCR product was purified and quantitated using the PSQ HS 96 Pyrosequencing System (Pyrosequencing, Inc.; Westborough, Mass.). The sequencing primer for pyrosequencing was (5′-AATAACTAAAATTACAAAC-3′) (SEQ ID NO: 6).

Methylation of the LINE-1 promoter was also investigated by a similar COBRA assay. A 50 μl PCR was carried out in 60 mM Tris-HCl pH=8.8, 15 mM Ammonium Sulfate, 0.5 mM MgCl₂, 1 mM dNTP mix, and 1 unit of Taq polymerase. 50 pmol of each PCR primer was used: 5′-TTGAGTTGTGGTGGGTTTTATTTAG-3′ (SEQ ID NO: 7) and 5′-TCATCTCACTAAAAAATACCAAACA-3′ (SEQ ID NO: 8). PCR cycling conditions were 95° C. for 30 seconds, 50° C. for 30 seconds, and 72° C. for 30 seconds for 35 cycles. The final PCR product was digested with the HinfI restriction enzyme. The digested PCR products were separated by electrophoresis on polyacrylamide gels.

### Example 4

### Cloning and Sequencing

PCR products were cloned using the TOPO-TA cloning kit (Stratagene) per the manufacturers protocol. Mini-preps were prepared using QIAprep Spin Miniprep Kit (Qiagen, Valencia, Calif.). The M.D. Anderson Cancer Center Sequencing Core Facility performed all DNA sequencing.

### Example 5

### PCR of Bisulfite Treated Alu Repetitive Elements Amplifies Multiple Unique Alu Repetitive Elements

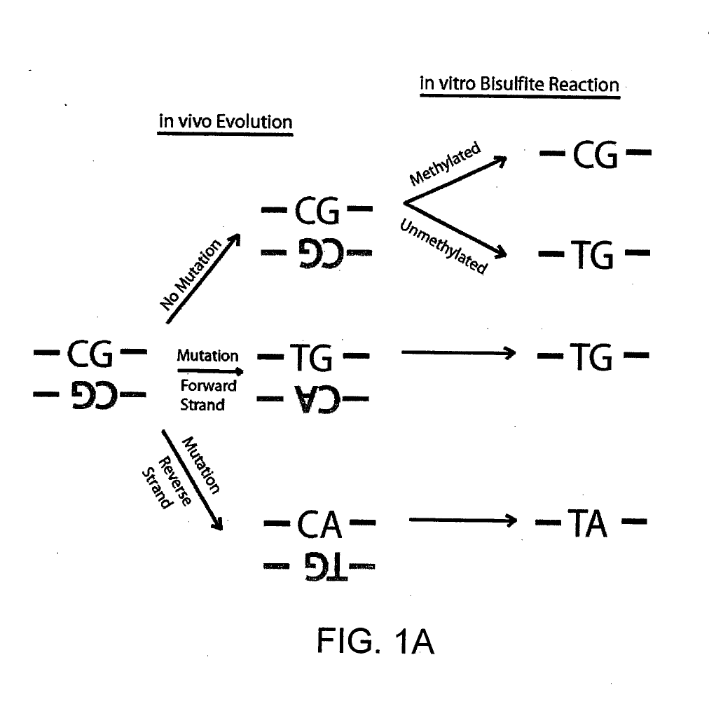

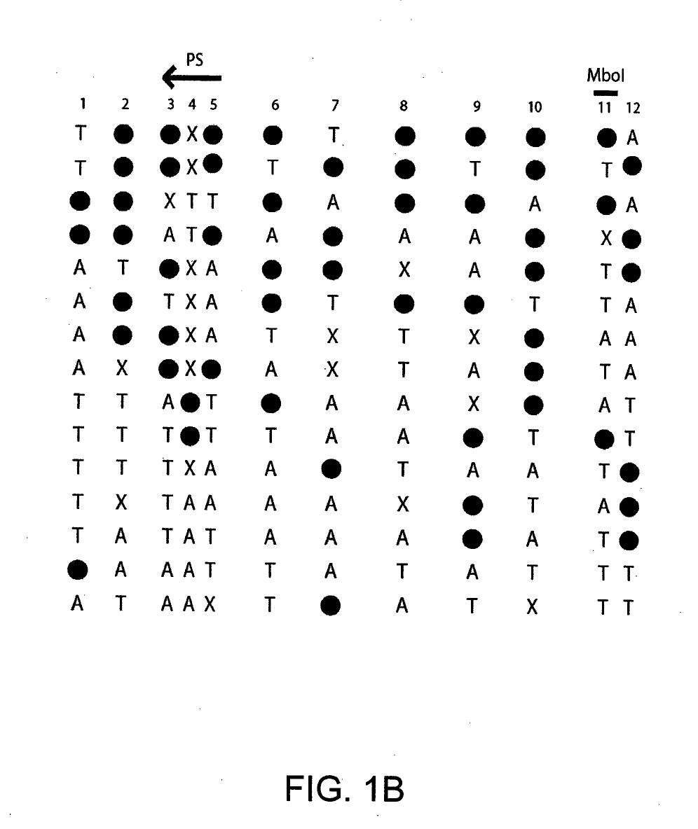

PCR primers were designed to amplify an approximately 150-base pair fragment of bisulfite treated DNA from Alu repetitive elements. The primers were designed to avoid potential CpG methylation sites in order to minimize amplification biases for methylated or unmethylated DNA. In order to assure that a pool of different Alu elements was being amplified from the genome the PCR product from 4 different human blood DNA samples was cloned using a TOPO TA-cloning kit (Invitrogen, Carlsbad, Calif.) and 60 individual clones representing individual PCR products were sequenced. The sequenced PCR products were all Alu elements, but all had unique sequences and no two clones showed an identical sequence. Representative data from one DNA sample is shown in FIG. 1.

### Example 6

### The Majority of Potential CpG Methylation Sites in Alu Elements are Mutated

Based on the consensus sequence of Alu elements it was estimated that the 150 base pair fragment should have 12 CpG sites. Therefore examining sequence data from 15 Alu element clones, one would expect potentially 180 CpG sites could be methylated. However, only 66 of 180 (36.7%) of the potential CpG sequences were maintained after bisulfite treatment and were therefore methylated. The remaining 114 CpG sites were either unmethylated or mutated either to TpG (53/180=24.4%), TpA (41/180=22.8%) or other mutations (20/180=11.1%).

Methylation could also be approximated by MboI digestion. In this analysis, restricted fragments are methylated while unrestricted fragments are either mutated or unmethylated. Analysis of normal human blood showed 27.2% methylation. The same DNA was treated with an excess of SssI methylase, which will methylate all CpG sites. Quantification of methylation of the SssI treated DNA by MboI digestion showed only 29.2% methylation. This indicates that 93% of potential CpG sites were methylated and the majority of CpG sites at the MboI recognition site had been mutated.

### Example 7

### Bisulfite Repetitive Element PCR Samples Several Thousand Repetitive Elements

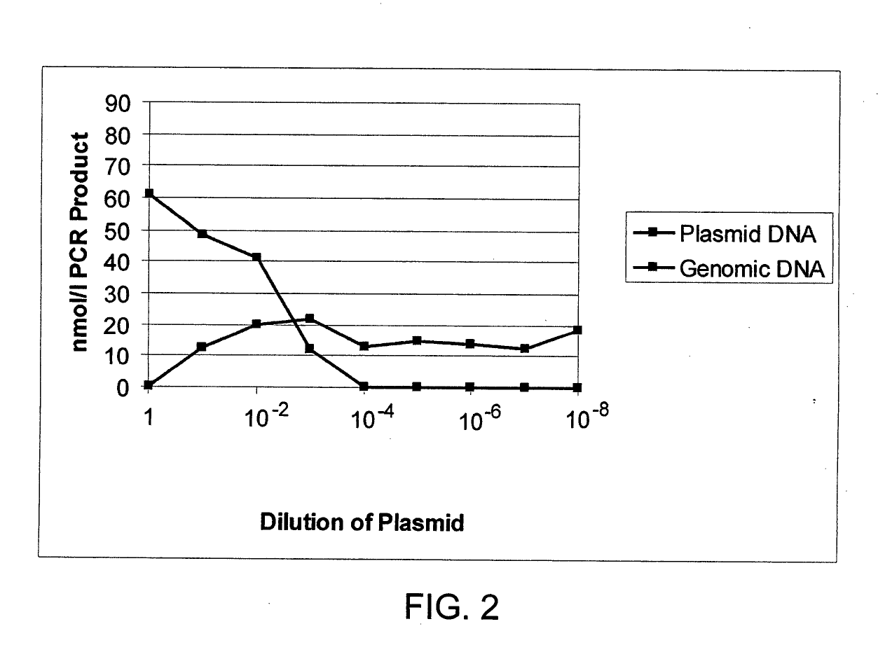

A competitive PCR experiment was performed where a fixed amount of bisulfite treated genomic DNA was mixed with serial dilutions of the plasmid containing the larger Alu element (FIG. 2). From these experiments, it was calculated that 25 ng of bisulfite genomic DNA gave equivalent PCR product as 0.5 ng of plasmid. With the assumptions that the human genome contains 3×10⁹ bases and 9×10¹¹ bases are in 1 ng of DNA, it was calculated that there were approximately 7,500 genome equivalents in 25 ng of DNA. The plasmid vector was 4.2 kilobases in size and contained one Alu element per plasmid. It was then calculated that 0.5 ng of plasmid contained 1×10⁸ Alu elements. Therefore a PCR reaction starting from 7500 genomes is equivalent to a PCR reaction starting from 1×10⁸ cloned Alu elements, implying that about 15,000 different Alu elements were being amplified from each genome. This diversity is bourne out by the fact that the cloned Alu elements in FIG. 1 were all unique.

### Example 8

### Bisulfite Repetititve Element PCR can Detect DNA Methylation Decreases in Cell Lines Treated with 5-aza-2′deoxycytidine

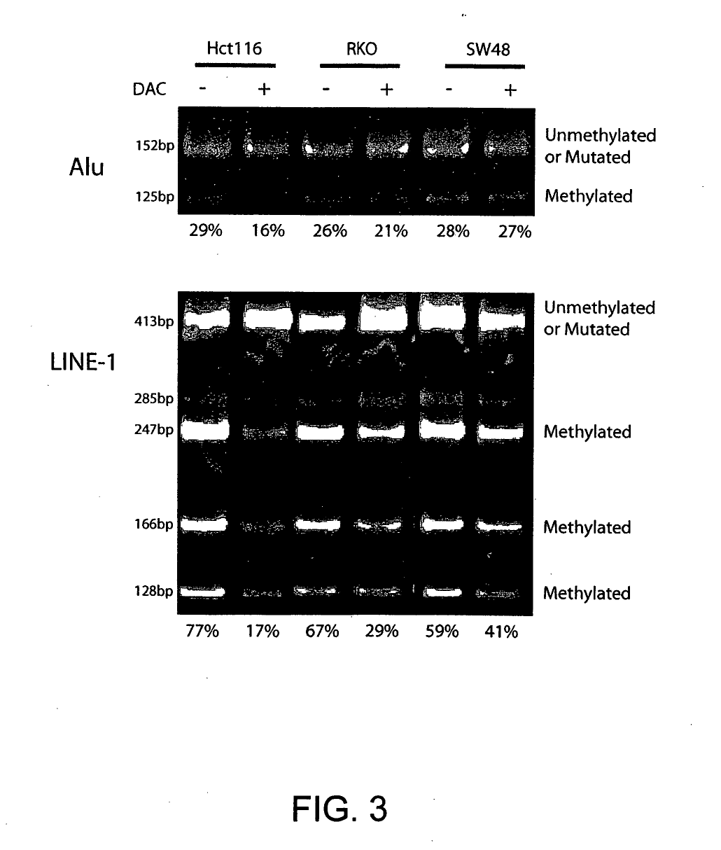

In order to test whether Alu element of LINE-1 methylation could approximate global methylation, COBRA, restriction digestion of the repetitive element PCR product, was used to quantitate DNA methylation in cell lines treated with 5-aza-2′deoxycytidine (DAC) (FIG. 3). The colon cancer cell line, Hct116, had 29% Alu element and 77% LINE-1 methylation prior to treatment, and 16% Alu element and 17% LINE-1 methylation after treatment with DAC. RKO cells treated with DAC showed a decrease from 26% to 21% in Alu element methylation, and a decrease from 67% to 29% in LINE-1 methylation. By contrast SW48 cells showed little change in methylation with DAC treatment with Alu methylation decreasing from 28% to 27% and LINE-1 methylation decreasing from 59% to 41%. Similar results were obtained for each cell line treated with DAC for both the Alu element and LINE-1 assays. In order to test the reproducibility of the assay, COBRA was used to measure Alu element methylation on the same DNA sample eight separate times and the standard deviation was only 2%. Thus both assays reliably detected inhibition of methylation induced by DAC. The Alu element assay showed about two thirds of the PCR product was not digested by MboI, however, this is not due Alu elements being largely unmethylated. As found by the sequencing data, the majority of Alu elements the CpG dinucleotide, and methylation target, had been mutated and was no longer a target for DNA methylation. Overall, these data show that repetitive element PCR can be used as a marker of global DNA methylation.

### Example 9

### Bisulfite Repetitive Element PCR can be Quantitated Using Pyrosequencing™

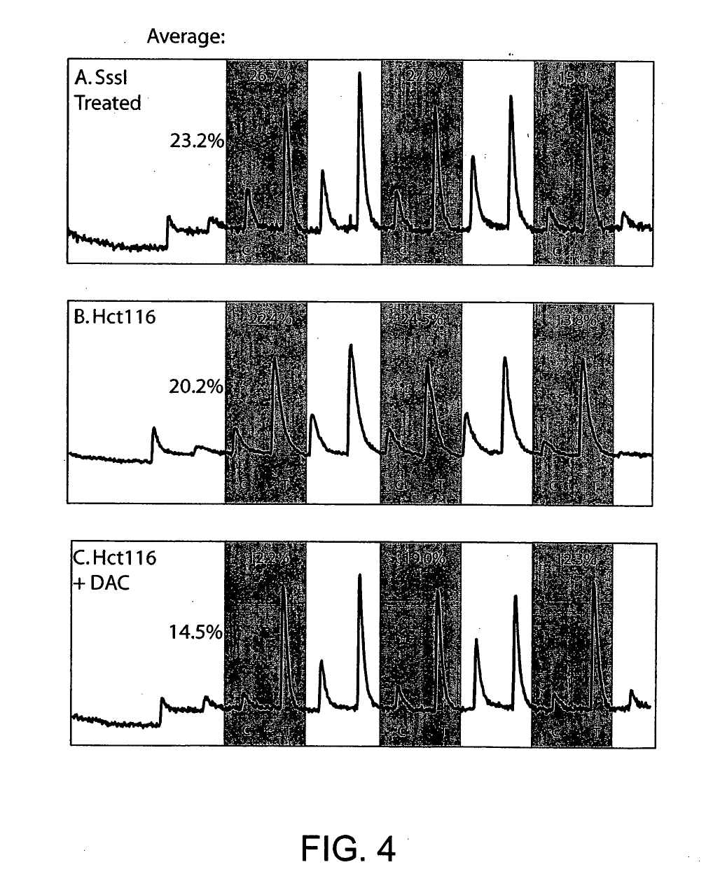

Bisulfite Alu PCR products could also be analyzed by pyrosequencing allowing for a rapid analysis of multiple CpG sites. Pyrosequencing is a direct sequencing by synthesis method originally developed to overcome artifacts of secondary structure and avoid gel electrophoresis. This method has the advantage of analyzing several methylation sites, is not restricted to restriction enzyme sites, avoids sequencing multiple clones, and allows accurate quantitation of multiple CpG methylation sites in the same reaction. Bisulfite Alu PCR products were pyrosequenced in an area that had three tandem CpG sites (FIGS. 1B and 4). This method was used to quantitate the decrease in methylation of the same Hct116 cells treated with 5-aza-2′deoxycytidine analyzed by the COBRA assay. In order to calculate the potential number of CpG sites that could be methylated, genomic DNA was double treated with an excess of SssI methylase, which will methylate all CpG sites, and bisulfite treated. By pyrosequencing only 23.2% of potential CpG sites could be methylated showing that most of the potential CpG sites had been mutated and could no longer be methylated (FIG. 4). Of these potential CpG sites 20.2% were methylated in Hct116 cells, or about 87% of the potential methylation sites. The difference between Alu methylation in genomic DNA and SssI methyltransferase treated genomic DNA was very small. This is consistent with previous results in which Alu elements were found to be 84.6% methylated in peripheral blood DNA (FIG. 1B). Treatment with DAC decreased methylation to 14.5%, or 62% of potential methylation sites. There was a small difference for methylation analysis of Hct116 cells using COBRA versus pyrosequencing. These differences are likely attributable to the fact that different CpG sites were analyzed (FIG. 1B) and by differences in the techniques.

### Example 10

### Methylation Changes in Normal and Tumor Colon Cancer

Normal colon mucosa and colon cancers were collected from consenting patients. Thirty-one matched pairs of normal colon mucosa and colon tumors were used in this study. An additional 9 normal colon samples were collected from patients undergoing colectomy for non-neoplastic reasons. DNA was isolated using standard phenol-chloroform extraction methods. DNA was bisulfite treated as described above. A previously described COBRA assay that examined Alu repetitive elements was developed as a global genome methylation assessment assay (Xiong and Laird, 1997). In brief, a 25 ul PCR reaction was carried out in 60 mM Tris-HCl pH 9.5, 15 mM Ammonium Sulfate, 5.5 mM MgCl2, 10% DMSO, 1 mM dNTP mix, 1 unit of Taq polymerase, 50 pmol of the forward primer (5′-GATCTTTTTATTAAAAATATAAAAATTAGT-3′) (SEQ ID NO: 1), 50 pmol of the reverse primer (5′-GATCCCAAACTAAAATACAATAA-3′) (SEQ ID NO:2), and approximately 50 ng of bisulfite treated genomic DNA. PCR cycling conditions were 96° C. for 90 seconds, 43° C. for 60 seconds, and 72° C. for 120 seconds for 27 cycles. After the PCR reaction was complete 15 μl of MboI buffer (New England Biolabs Buffer #3) were added along with 5 units of MboI Restriction Enzyme and water to a final volume of 150 ul. The restriction digestion was incubated at 37° C. overnight to assure complete digestion. The digested PCR product was then precipitated by adding 1 μl glycogen and 2 volumes of ethanol and kept at −20° C. for at least 4 hours. Samples were then centrifuged for 30 minutes, the ethanol was poured off and the pellet was air dried prior to resuspending in a small volume (5-10 μl) of water. The digested PCR products were then run on an Agilent Biosystems DNA analyzer to quantitate the cut (methylated) and uncut (methylated) DNA. Each sample was run at least twice and the data reported is an average of all runs.

The methylation sites in Alu elements are frequently mutated from C to T by spontaneous deamination, which results in a gross underestimating of the amount of 5-methylcytosine. Therefore, control genomic DNA was double treated with SssI methylase and bisulfite treated to act as a 100% methylated control. Alu methylation is reported as a percentage of this fully methylated control standard.

Microsoft Excel 2000 was used to analyze the data. To determine the relationship of Alu element methylation and age, linear regression statistics were used. A two-sided t-test was used to compare Alu methylation in patients 50 years old and younger to patients older than 50 years of age.

### Example 11

### Determination of the Percentage of Alu Methylation

Genomic DNA was isolated, bisulfite treated and a PCR was performed. The PCR primers were designed from an Alu consensus sequence that allowed the primers to theoretically amplify most Alu elements in the genome. Previous studies have shown that at least 15,000 different Alu elements are being sampled by this assay. COBRA (Combined Bisulfite Restriction Analysis) was used to quantitate the methylation of a single CpG site within Alu elements. On the whole, only 20.9 to 28.7% of Alu elements were methylated at this site. However, the COBRA assay cannot distinguish between sites that are unmethylated from sites where the CpG site was mutated and no longer a target for DNA methylation. In order to determine the number of methylatable sites, genomic DNA was treated with an excess of SssI methylase. Analysis of this DNA showed that a maximum of 29.6% of sites could be methylated. This indicates that over 70% of the studied CpG sites in Alu elements have been mutated which is consistent with previous reports that CpG sites are highly mutagenic. The Alu methylation in this paper is therefore reported as a corrected percentage to account for this.

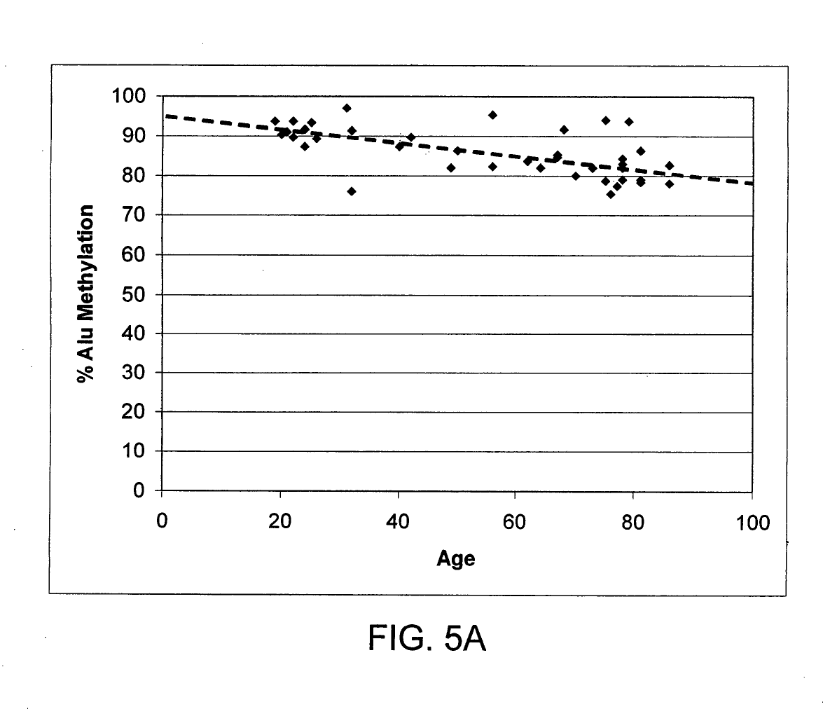

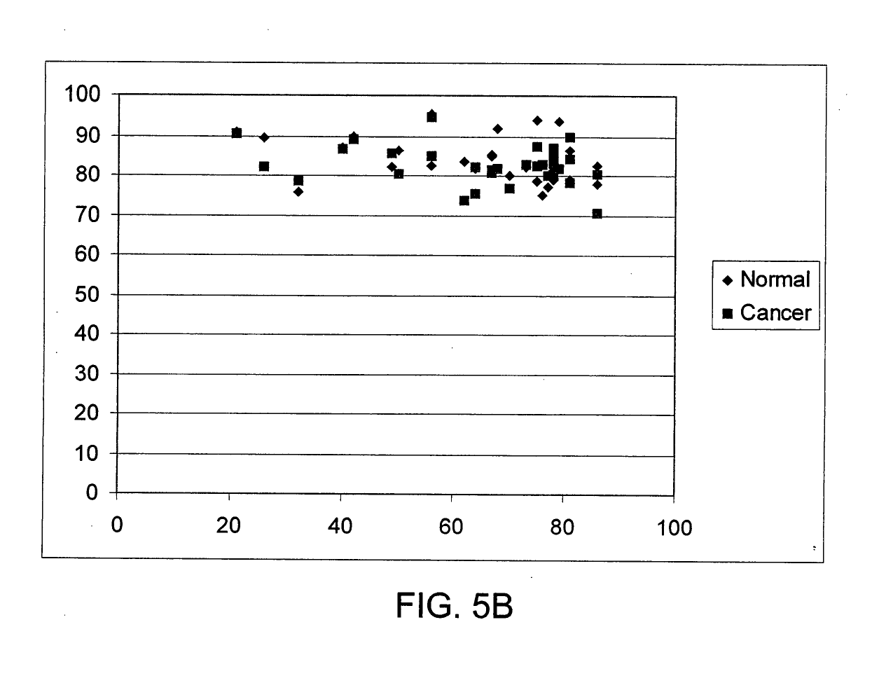

Quantitative analysis of Alu element DNA methylation in 40 normal and 31 colon cancers was performed. Methylation of Alu elements overall appeared to be very heavy with methylation ranging from 70.5 to 97.0% of methylatable sites. Mean methylation was 85.7% and 82.6% for normal colon and colon cancers respectively. Colon samples were studied from patients between 19 to 86 years of age. Plotting the amount of Alu methylation versus age in years shows there is a progressive decline in Alu element methylation with increasing age that is statistically significant (R=0.59, p=0.00007) (FIG. 5). Regression analysis of Alu element methylation in normal colonic mucosa and an assumption of zero-order decay of Alu element methylation allows us to derive a formula to estimate Alu element methylation at any age.
Normal Colon Alu Methylation %=94.2−(0.15*Age in years)

From this formula one can estimate that Alu element methylation is 94.2% at birth and decays at a rate of 0.15% per year. Another way of analyzing the data was to divide the patients in to those 50 years of age and younger versus those over 50 years of age. This age was chosen arbitrarily as the age at which colon cancer screening begins with sigmoidoscopy or colonoscopy. The mean Alu methylation in patients 50 years of age and under was 89.3% (n=16) while those over 50 years of age was 83.3% (n=24) (p=0.0005 two-sided t-test). In a comparison of these two groups by a two-sided t-test they difference in methylation was highly significant with a p=0.0005.

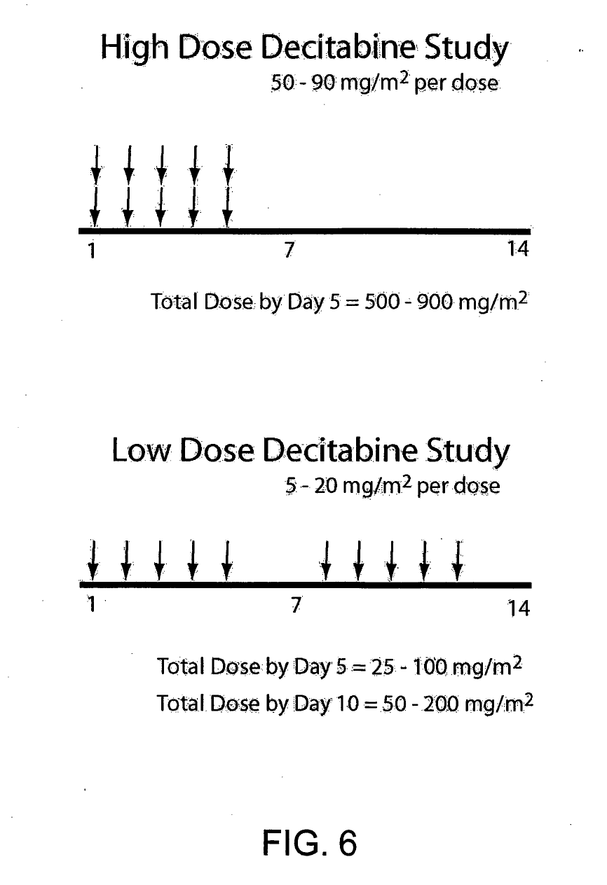

Thirty-one of the normal colon mucosa samples analyzed were matched sets with colon cancer samples. There were no age related changes in Alu element methylation in colon cancer (FIG. 6). Comparison of colon cancer to matched normal colon mucosa from the same patient showed no consistent changes in Alu element methylation. There was a mean decrease in Alu methylation of only 1.3% from normal to colon, but the change in methylation varied from a 12% decrease from normal to cancer to a 7.4% increase from normal to cancer. About half, 17 of 31 patients showed a decrease in Alu methylation from normal to cancer while 14 of 31 patients showed an increase.

Clinical history was available on 25 of the patients studied. There was a statistically significant decrease in Alu methylation depending on lymph node involvement. Patients with stage II colon cancer (n=14), localized disease without lymph node or distal metastases, had a mean Alu element methylation of 85.1% (SEM=1.4%) in their normal colon with their matched cancers having a mean Alu methylation of 84.9% (SEM=1.1%). Patients with stage III (n=8) and stage IV (n=3) had a mean Alu element methylation of 83.4% (SEM=1.5%) in their normal colon mucosa, but only 79.7% (SEM=1.7%) Alu methylation in their cancers. Thus patients with later stage disease appeared to have a statistically significant decrease (p=0.016) in Alu element methylation in their tumors, but not their uninvolved mucosa. Further analysis of samples by patient gender and location (left vs. right) showed no correlation to the degree of Alu element methylation. No clinical outcome or therapy reponse data were available on these patients.

### Example 12

### Decrease in Alu Element Methylation with Age

A decrease in Alu element methylation with age was observed. In normal colon mucosa from patients 50 years of age and younger mean Alu element methylation was 89.5% (SEM=1.6%, N=13, Average Age=29 years) while in those patients over 70 years of age Alu element methylation was 81.9% (SEM=1.3%, N=14, Average Age=79). This decrease in methylation is small, only 7.5%, over a fifty year period. Thus, in human neoplasia, the actual hypomethylation observed is at least one order of magnitude lower than that in the described mouse models.

### Example 13

### Hypomethylation and Chromosomal Instability

A cell line panel was analyzed for differences in Alu element and LINE-1 methylation. Alu methylation averaged 83.1% (SEM=1.4%) in stable cell lines (Hct116, LoVo, SW48, RKO, DLD1) vs. 78.9% (SEM=5.2%) in unstable cell lines (HT29, SW480, SW837, Colo205) (FIG. 1A). LINE-1 methylation in stable vs. unstable cell lines was 56.6% (SEM=6.77) and 50.6% (SEM=8.76), respectively. Thus there was only small decrease in CIN− vs. CIN+ cell lines.

### Example 14

### Methylation Changes in Leukemia

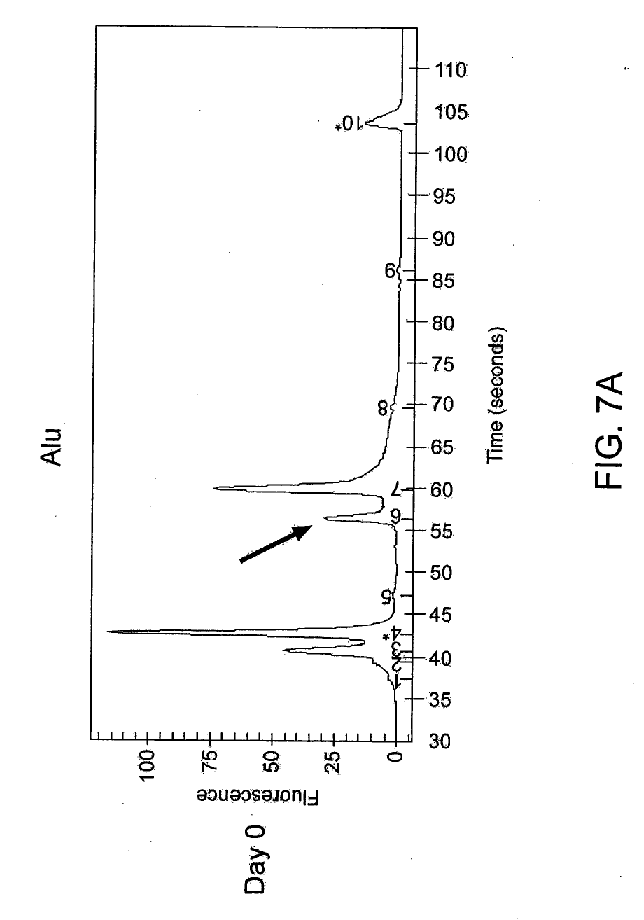

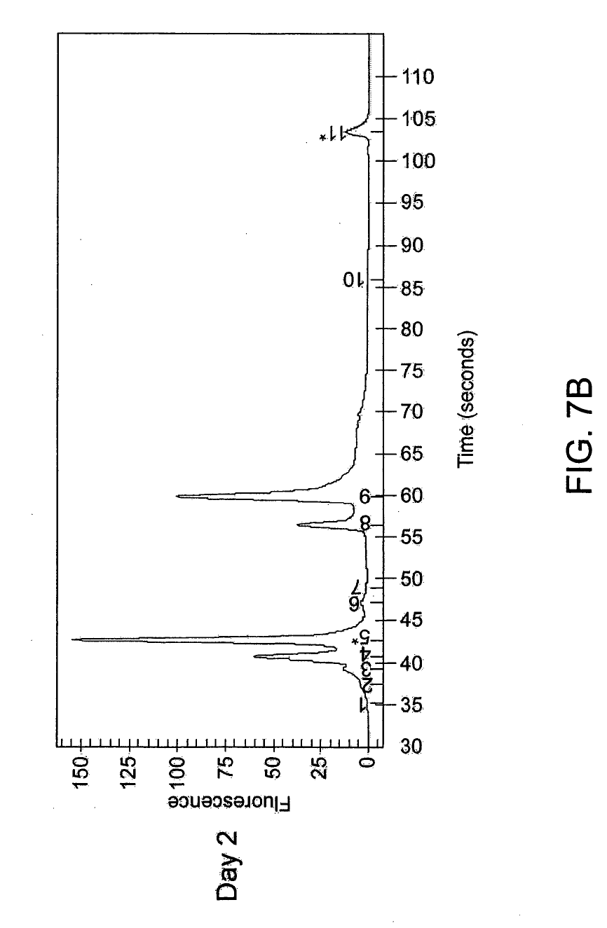

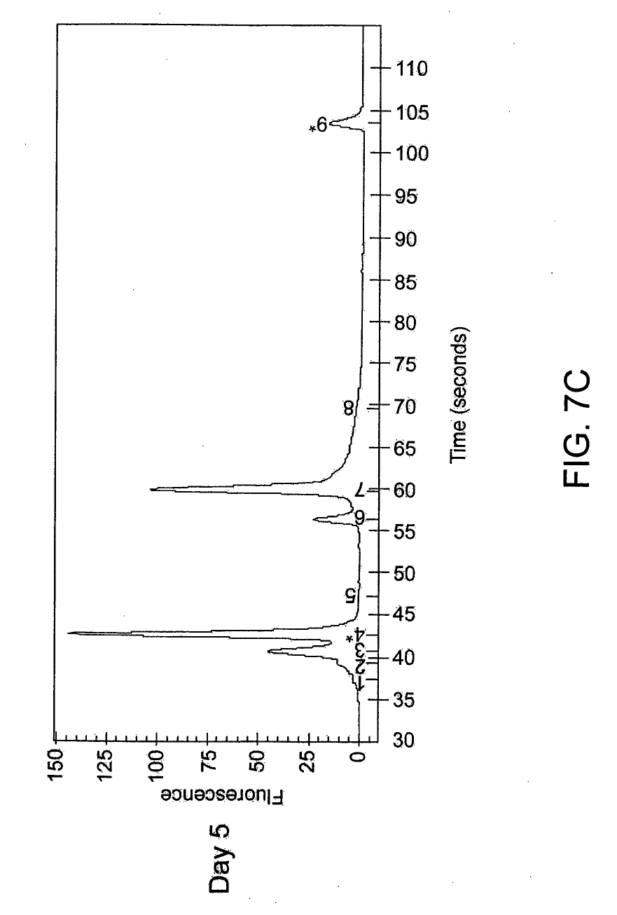

The Alu element assay was used to measure demethylation induced by this drug in 41 patients with leukemia treated at different doses. As shown in FIG. 7, 5-aza resulted in an average Alu demethylation of 7.7% (SEM=1.68, SD=9.82%) 4 to 7 days after treatment with the 31.5% being the maximum decrease seen in a single patient. In addition methylation had returned to baseline in as little as 15 days of finishing treatment. Pharmacologically induced demethylation appears to be very transient with methylation returning to pretreatment levels within two weeks of being off therapy.

Patients with CML were treated with the hypomethylating agent 5-aza-2′-deoxycytidine, and methylation was measured by the LINE assay on peripheral blood mononuclear cells. Tables 1-3 show the results in three patients, indicating decreases in methylation (to a variable extent) in each one. Day indicates the day after the first dose of 5-aza-2′-deoxycytidine (which is given on days 1-5 and 7-12 of each cycle). Cycle indicates the beginning (and cycle #) of each course of 5-aza-2′-deoxycytidine.

TABLE 1
Patient 1

| | Methylation | |
|---|---|---|
| Day | (%) | Cycle |
| 1 | 80.7 | 1 |
| 12 | 59.3 | |
| 17 | 64 | |
| 37 | 69.2 | 2 |
| 40 | 81.3 | |
| 43 | 57.7 | |
| 50 | 70.6 | |
| 73 | 76.1 | |
| 74 | 73.9 | 3 |
| 79 | 66.9 | |
| 113 | 71.1 | 4 |

TABLE 2
Patient 2

| | Methylation | |
|---|---|---|
| Day | (%) | Cycle |
| 1 | 74 | 1 |
| 5 | 61.9 | |
| 12 | 50.2 | |
| 46 | 74.8 | 2 |
| 50 | 61.3 | |
| 57 | 66.6 | |
| 115 | 71 | 3 |
| 120 | 67.4 | |
| 122 | 64.9 | |
| 123 | 53.6 | |
| 127 | 59.6 | |
| 183 | 73.3 | 4 |
| 187 | 60.8 | |
| 194 | 53.5 | |
| 226 | 79.6 | |

TABLE 3
Patient 3

| | Methylation | |
|---|---|---|
| Day | (%) | Cycle |
| 1 | 76.1 | 1 |
| 5 | 55 | |
| 12 | 63.4 | |
| 44 | 76.1 | 2 |
| 50 | 53.8 | |
| 54 | 48.2 | |
| 78 | 69.9 | |
| 83 | 67.3 | |

### Example 15

### Decitabine Study

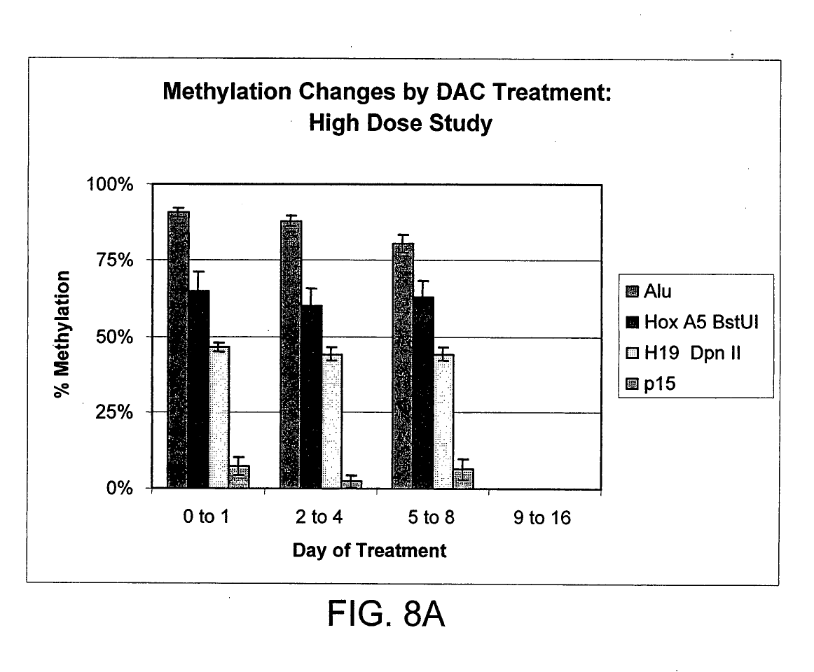

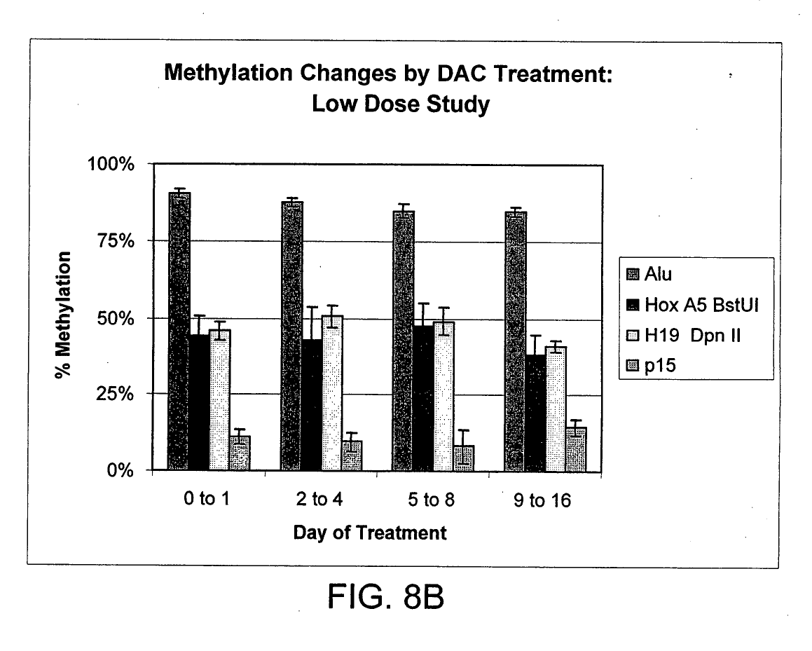

Patient samples were collected from patients treated as part of two clinical studies using 5-aza-2′deoxycytidine (decitabine) as a single agent. The first study treated patients with 50-90 mg/m2 of decitabine twice daily for 5 consecutive days (See FIG. 8). The second study was a phase I study which attempted to capitalize on the demethylating properties of decitabine by using a lower dose of decitabine, 5-20 mg/m² once daily, for a longer period of time, 10 days over a two week period (See FIG. 8). Peripheral blood samples were collected prior to, during and after treatment. Donation of blood samples for laboratory studies was voluntary and 143 samples were collected from 41 patients participating in both studies (See Table 4). Due to competing protocols and the availability of patients at the time of the two respective studies the majority of the patients enrolled on DM−, the “High-Dose” study were CML patients and the majority of the patients enrolled on DM−, the “Low-Dose” study were AML or High Risk MDS patients.

Table 4: Samples Studied

Donation of peripheral blood for laboratory analysis was voluntary, and only some of the patients enrolled on the two studies participated. A total of 134 samples were collected for analysis (52 in the High Dose Study and 82 in the Low Dose Study). The majority of patients treated in the High Dose Study were CML patients, however, the majority of patients treated in the Low Dose Study were AML or High risk MDS patients.

| | | | | | Response | |
|---|---|---|---|---|---|---|
| | Total | Diagnosis | | Treatment | No | Response |
| | Patients | AML/MDS | CML/CMML | CR/PR | Response | Rate |
| High Dose Study | 18 | 3 | 15 | 7 | 11 | 39% |
| Low Dose Study | 23 | 20 | 3 | 7 | 13 | 35% |
| Total | 41 | 23 | 18 | 14 | 24 | 34% |

### Example 16

### Analysis of LINE-1 and p15 Methylation by Bisulfite PCR and Pyrosequencing

DNA was isolated from peripheral blood samples using standard phenol-chloroform extraction methods. DNA was bisulfite treated as described above. Bisulfite treated DNA was stored at −20° C. until ready for PCR amplification and analysis by direct sequencing, restriction digestion (COBRA), or pyrosequencing.

The LINE-1 assay was based on a similar principle Alu element COBRA assay, but used non-selective PCR of Long Interspersed Nucleotide Elements and pyrosequencing to quantiate methylation. A 50 μl PCR was carried out in 60 mM Tris-HCl pH=8.8, 15 mM Ammonium Sulfate, 0.5 mM MgCl₂, 1 mM dNTP mix, and 1 unit of Taq polymerase. 10 pmol of each PCR primer was used: 5′-TTGAGTTGTGGTGGGTTTTATTTAG-3′ (SEQ ID NO: 7) and 5′-TCATCTCACTAAAAAATACCAAACA-3′ (SEQ ID NO: 8), and 1 pmol of a universal biotinylated primer (5′-GGGACACCGCTGATCGTATA-3′) (SEQ ID NO: 5). PCR cycling conditions were 95° C. for 30 seconds, 50° C. for 30 seconds, and 72° C. for 30 seconds for 35 cycles. The PCR product was purified and quantitated using the PSQ HS 96 Pyrosequencing System (Pyrosequencing, Inc.; Westborough, Mass.). The sequencing primer for pyrosequencing was (5′-AATAACTAAAATTACAAAC-3′) (SEQ ID NO: 6).

### Example 17

### Decitabine Reduces Total Genomic 5-methylcytosine and Induces Global Hypomethylation of Repetitive DNA Elements

In order to see if decitabine could inhibit methylation over the entire genome we quantitatively measured the methylation of Alu repetitive elements, LINE repetitive elements and total 5-methylcytosine content of patient samples collected before, during and after treatment with decitabine.

Examination of Alu repetitive elements seemed to have several advantages over the gene specific loci examined. Alu elements are more abundant and more heavily methylated in the genome. PCR reactions were easier to perform because of the multiple copies of this repetitive DNA target. In addition Alu elements were found to be very heavily methylated in the human DNA with 90.5% of methylatable sites being methylated prior to decitabine treatment this allowed for small decreases of DNA methylation to be detected. Analysis of Alu elements was superior to gene specific methylation analysis for this reason and therefore was used for the remainder of this study. Overall most patients, 88%, showed a decrease in Alu methylation with decitabine treatment, although 12% of patients did show a paradoxical increase in DNA methylation with decitabine treamtment. The mean decrease in Alu methylation was only 7.7% with a range of −10.3 to 31.5% (SD=8.2%).

In order to confirm these results LINE repetitive elements were examined using a pyrosequencing based assay to DNA quantitate methylation. These data were confirmed by direct measurement of 5-methylcytosine in the genome. DNA was isolated from peripheral blood specimens and single nucleotides were quantitated using liquid-chromatography mass spectroscopy.

### Example 18

### Dose Dependence of Decitabine Inhibition of DNA Methylation

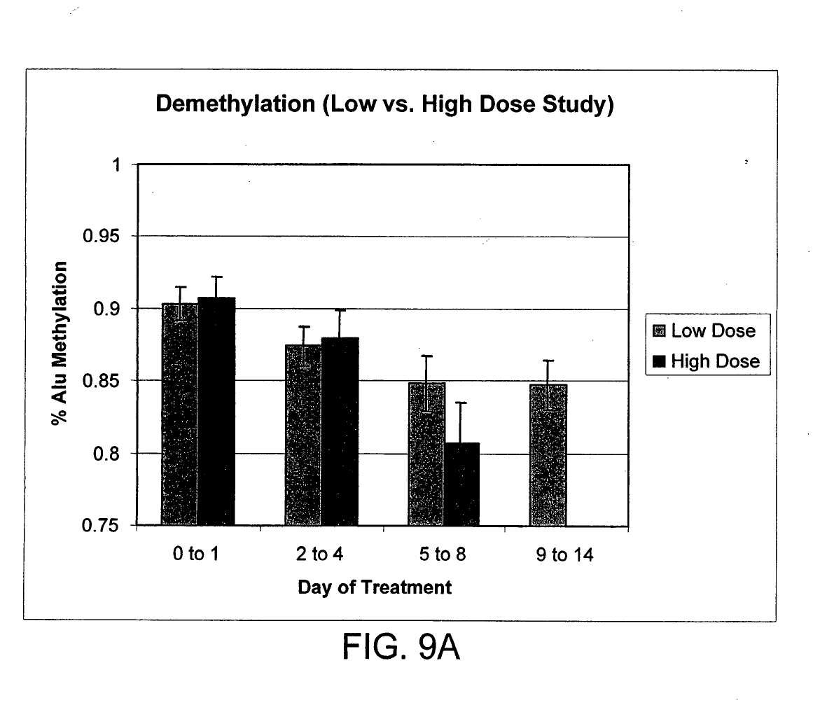

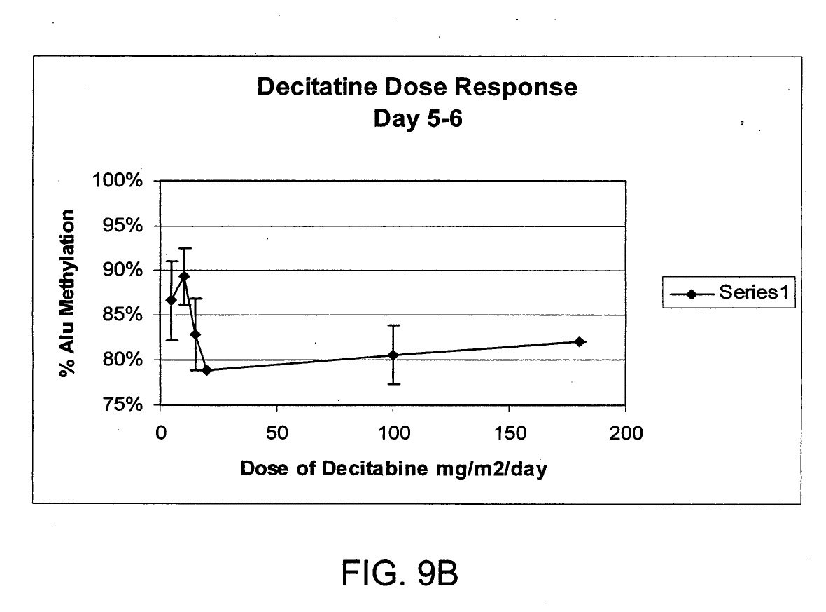

Demethylation induced by decitabine was dose dependent. This was examined in two ways. Comparison of the High Dose study to the Low dose study showed that the mean degree of demethylation induced by decitabine was greater for the higher dose study (FIG. 9A). Demethylation could be detected as early as day 2 for patients treated with either dosing regimen. By day 5 to 8 patients treated on the High Dose study showed a 10% mean decrease of Alu element methylation whereas those patients treated on the Low dose study showed only a half of that with a 5% decrease in methylation. Interestingly in the Low dose study additional days of treatment beyond day 8 with decitabine did not seem to further decrease the methylation of Alu elements. The demethylation in patients treated with low dose decitabine seemed to plateau at 5-8 days, after the first week of treatment. Interestingly the patients treated at higher doses seemed to have a lower mean methylation below the plateau.

Another way to examine the dose dependence of decitabine induced demethylation was to pool the patients treated in both studies. The Low Dose study was a 3+3 phase I clinical trial in which 3 patients each were treated at the dose level 5, 10, 15, and 20 mg/m2/day. We compared day 5-6 of these patient samples to day 5-6 of the phase II High Dose Study. The high dose study treated patients initially at 90 mg/m2 twice daily (180 mg/m2/day), but the study was later amended to treat patients at 50 mg/m2 twice daily (100 mg/m2/day). This allowed us to Patient were treated at six dose levels of decitabine (5, 10, 15, 20, 100 and 180 mg/m2/day) at day 5-6 which was the last day of treatment for the High dose study. Again more demethylation was seen at higher doses of decitabine treatment, however, there did not appear to be a significant increase in demethylation beyond 20 mg/m2/day.

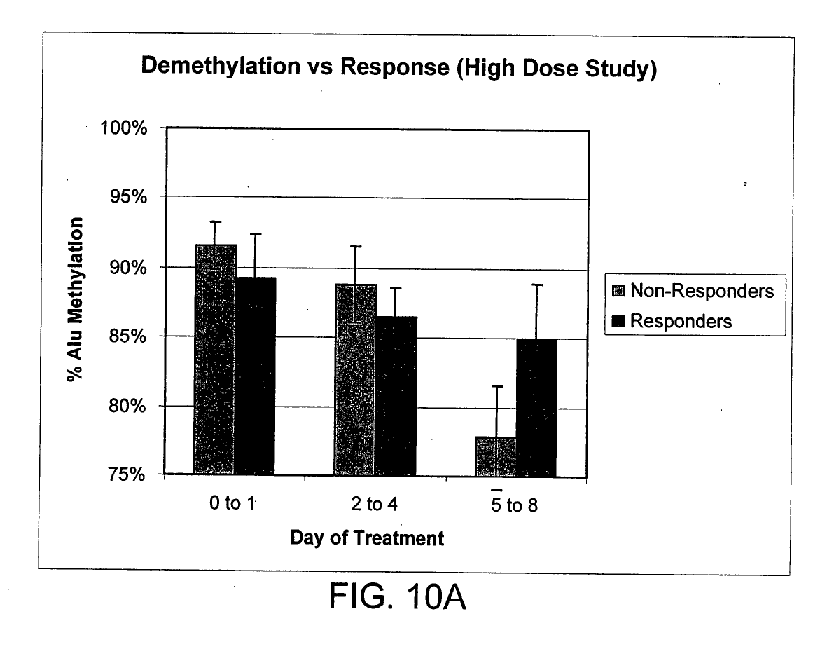

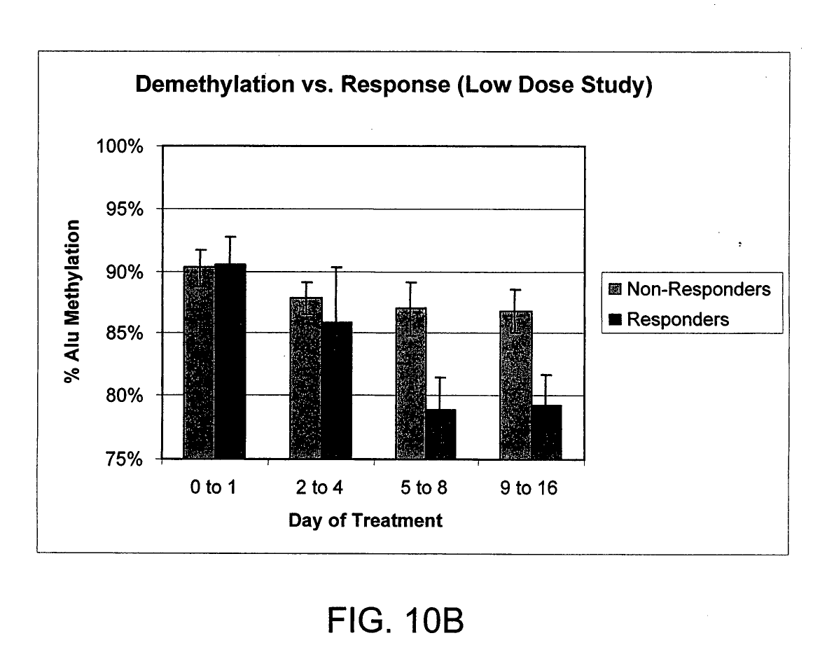

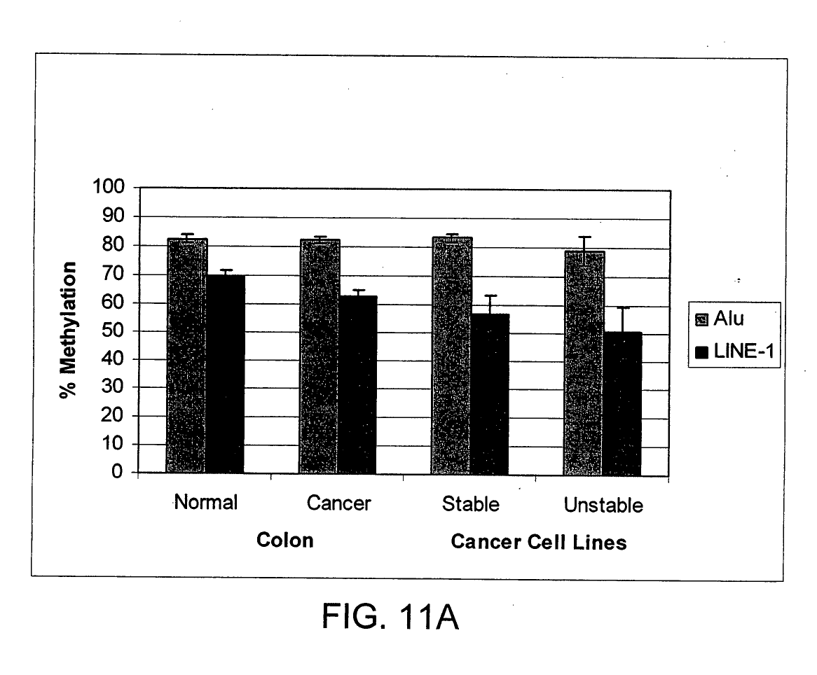

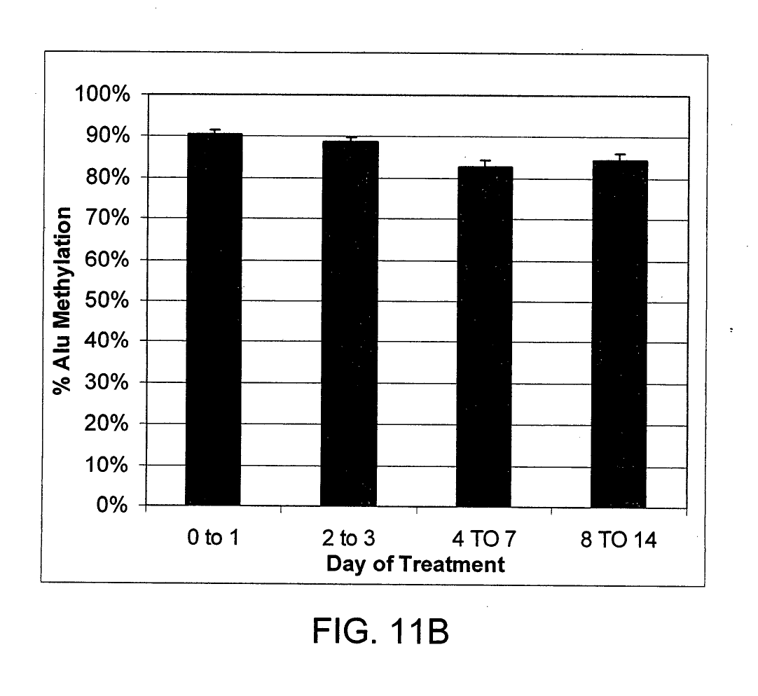

Analysis of Alu element methylation did delineate two key features between the High dose study and the Low dose study. In the High dose study both responders and non-responders to decitabine did show a mean decrease in methylation (FIG. 10A). Interestingly the non-responders showed more demethylation than those patients whose leukemia did respond to decitabine therapy. This difference was not statistically significant (p=0.23) by a two-sided t-test.

In contrast the Low dose study patients also showed decreases in methylation in both the non-responders and responders, but in this case the responders showed a statistically significant decrease by days 5-8 and on days 9-14 (p=0.04 and p=0.02 respectively) when compared to non-responders in a two-sided t-test. This paradoxical finding between the decrease in methylation and the clinical benefit of decitabine may be attributable to the disease, predominately CML in the High dose study versus AML in the Low Dose study. A more attractive possibility is that the actual dosing regimens may be crucial to how decitabine has clinical activity. In the high dose study decitabine was given at high doses, and may act as a cytotoxic pyrimidine analog, whereas in the Low dose study decitabine was given at low doses for longer periods of time to take advantage of its demethylating properties.

### Example 19

### LINE-1 Methylation Assay in Normal and Cancer Cell Lines

Nineteen tumor specimens were obtained from patients diagnosed with HNSCC at the Department of Surgery, School of Medicine of São Jose do Rio Preto, São Paulo, Brazil and Department of Head & Neck and Skull Base Surgery, Arnaldo Vieira de Carvalho Hospital, Sao Paulo, Brazil. Also, 88 tumor specimens and apparently normal tissue adjacent to tumor were obtained from patients diagnosed with colorectal carcinomas treated at the University of Texas M. D. Anderson Cancer Center (Houston, Tex.). Peripheral blood lymphocytes and normal oral mucosa swabs were obtained from six healthy individuals. Cancer cell lines from colon (RKO, SW48, SW480, Hctl 16, DLD-I, HT-29, LoVo), breast (MB-435), lung (NCI-H249, Hut64), leukemia (HL-60) and liver (HepG2) were obtained from the American Type Culture Collection. All of these cells were grown at 37° C. in a humidified atmosphere composed of 95% air and 5% CO₂ in a monolayer culture consisting of a 1:1 (v/v) mixture of Dulbecco's modified Eagles's medium, 10% regular fetal bovine serum, antibiotics, and either Ham's F-12 nutrient mixture or RPMI 1640 medium. DNA from all specimens and cell lines were obtained by treatment with proteinase K and phenol-chloroform extraction.

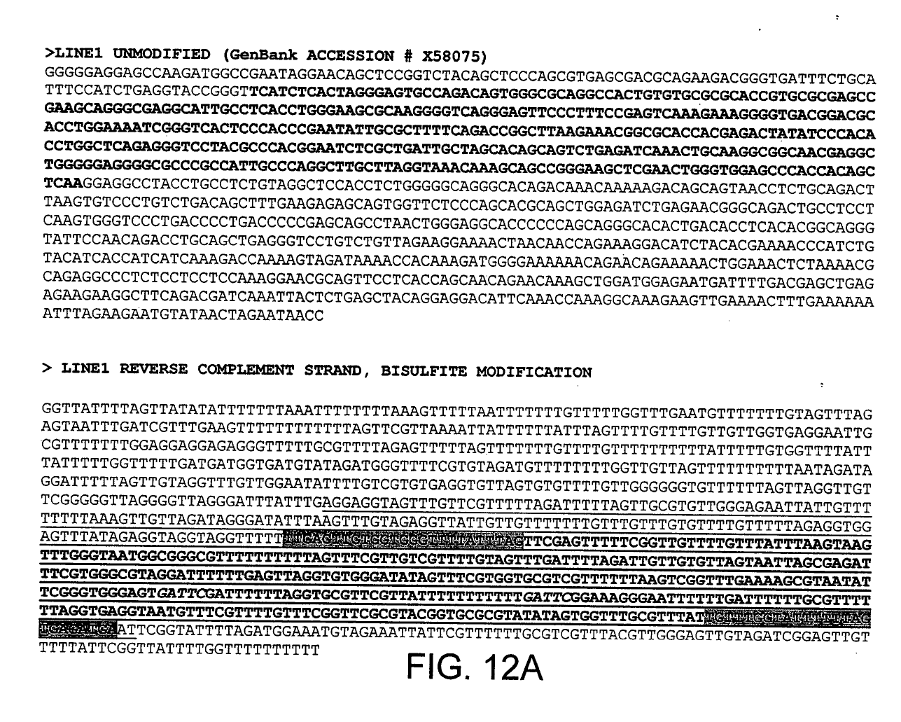

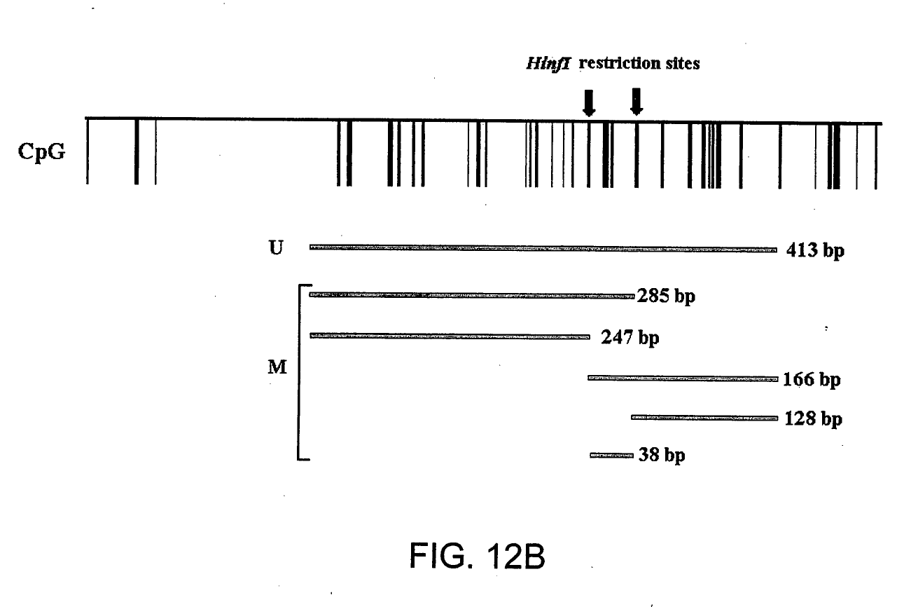

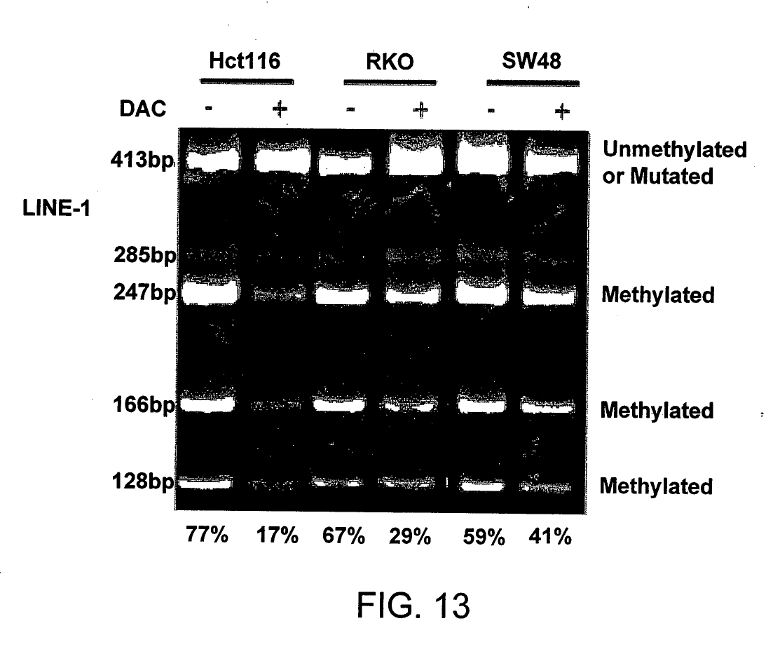

Methylation of the L1 promotor was investigated by COBRA assay (Xiong and Laird, 1997), and bisulfite treatment was performed as described above. A 50 ml PCR was carried out in 60 mM Tris-HCl pH=8.8, 15 mM Ammonium Sulfate, 0.5 mM MgCl2, 1 mM dNTP mix, and 1 unit of Taq polymerase. PCR cycling conditions were 95° C. for 30 seconds, 50° C. for 30 seconds, and 72° C. for 30 seconds for 28 cycles. 50 pmol of each PCR primer was used L1/F 5′-TTGAGTTGTGGTGGGTTTTATTTAG-3′ (SEQ ID NO:7) and L1/R 5′-TCATCTCACTAAAAAATACCAAACA-3′ (SEQ ID NO:8) to amplify fragments of 413 bp, which were cleaved into fragments of 285 bp, 247 bp, 166 bp, 128 bp and 38 bp by digestion with HinfI restriction enzyme. The LINE-1 sequence before and after bisulfite modification with the primer positions and the restriction map of the LINE-1 promoter are presented in the FIG. 12. Digested PCR products were separated by electrophoresis on 6% polyacrylamide gels. Gels were stained with ethidium bromide, imaged, and quantitated in a Bio-Rad Geldoc 2000 imager (Bio-Rad, Hercules, Calif.). The methylation density for each sample was computed as a ratio of the density of the digested band to the density of all bands in a given lane.

### Example 20

### LINE-1 Assay Results in Normal Tissues And Cancer Cell Lines

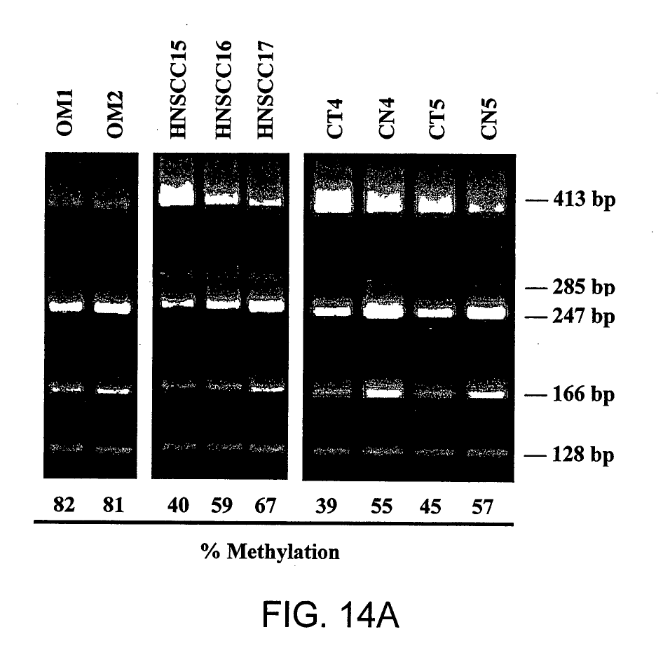

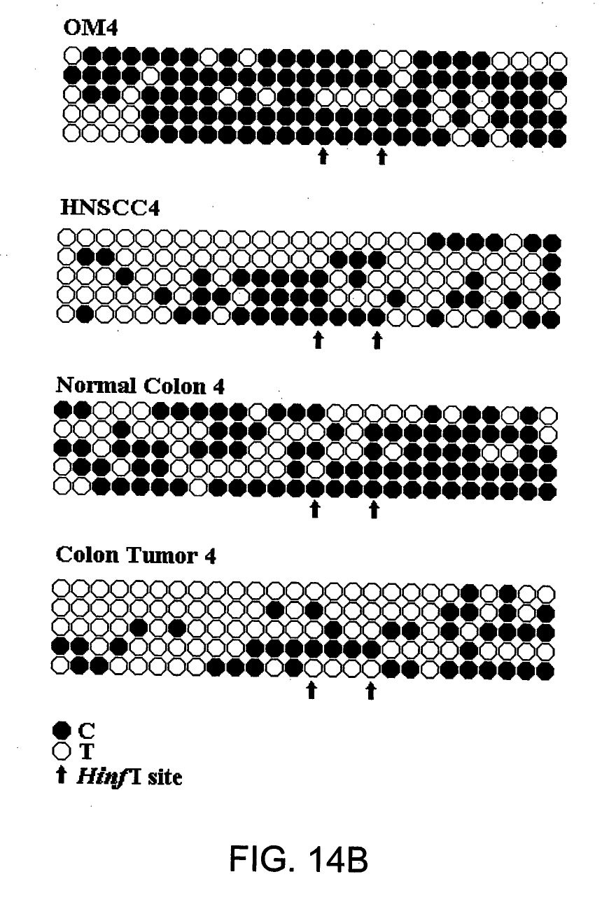

The LINE-1 methylation assay indicated the methylation density in normal oral mucosa keratinocytes and the density of these retrotransposons in normal tissues. An average of 79% was found after analysis of 6 samples. When the assay was applied to both head and neck tumor samples and normal and tumor colon pairs, we found lower degree of methylation in these tissues compared to PBL samples, with a more intense demethylation in tumor tissues (P<0.0001). LINE-1 methylation in cell lines treated with the demethylating agent 5-aza-2′deoxycytidine (DAC) was investigated. As presented in the FIG. 14, the DAC treated cell lines exhibited a lower degree of methylation as expect from the well-known effect of this drug in vitro.

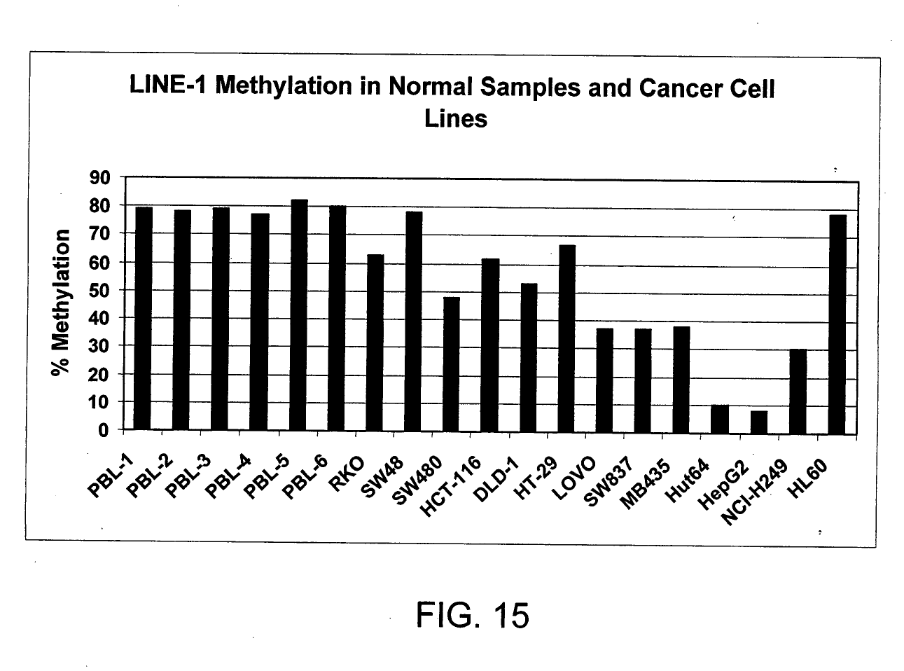

Human peripheral blood lymphocytes (PBL) were found to have an 80% methylation density while cell lines presented hypomethylation of LINE-1 were found to have a 90% methylation density (more than 10%) (FIG. 15). These findings corroborate the description of LINE-1 hypomethylation in cancer and its application as a tumor marker.

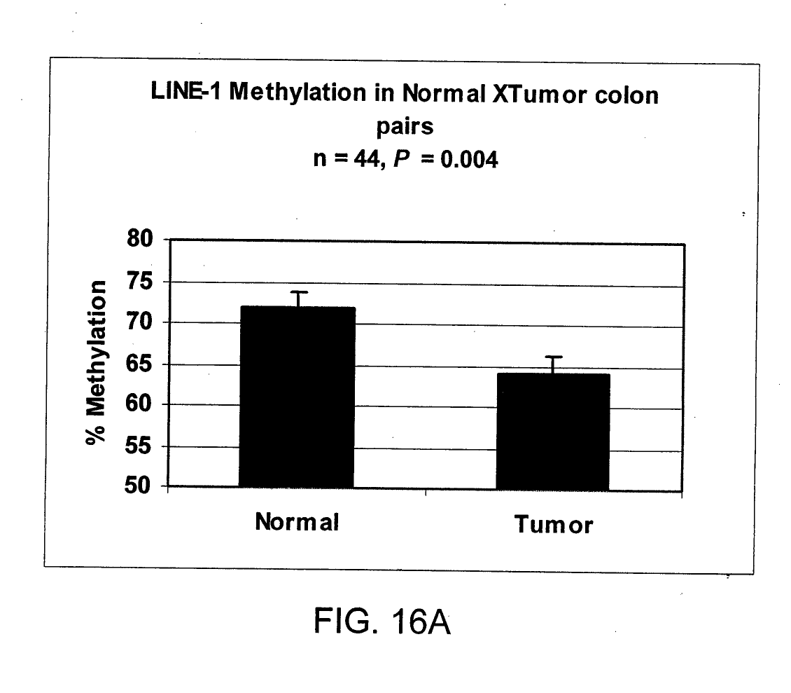

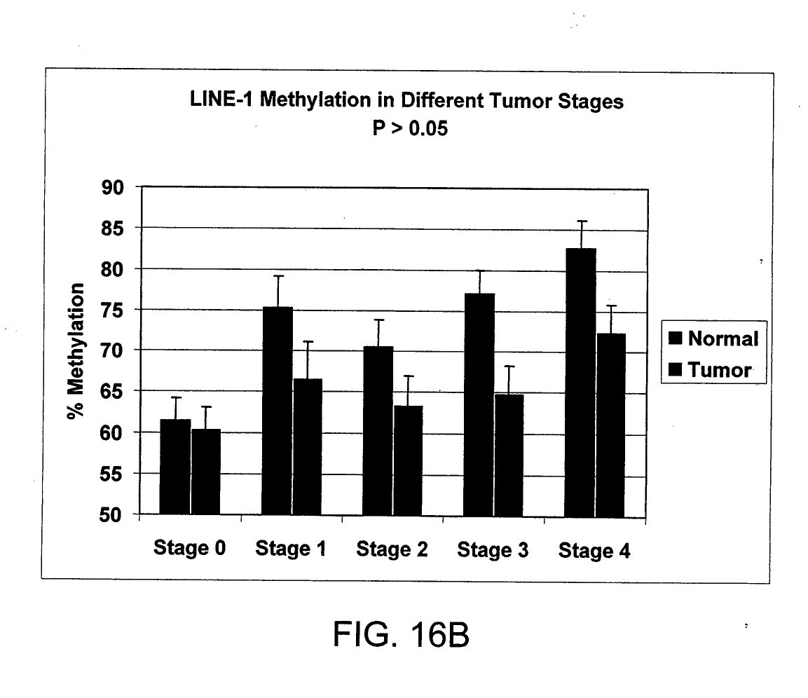

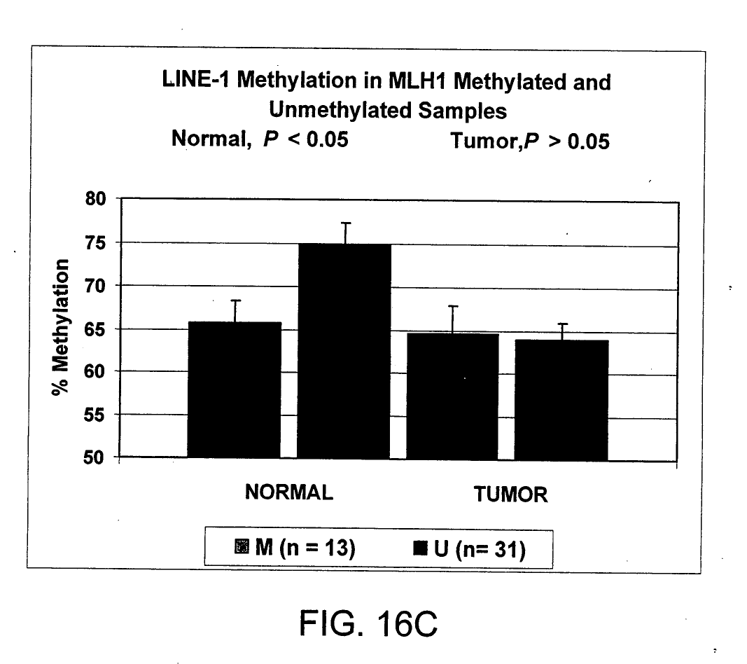

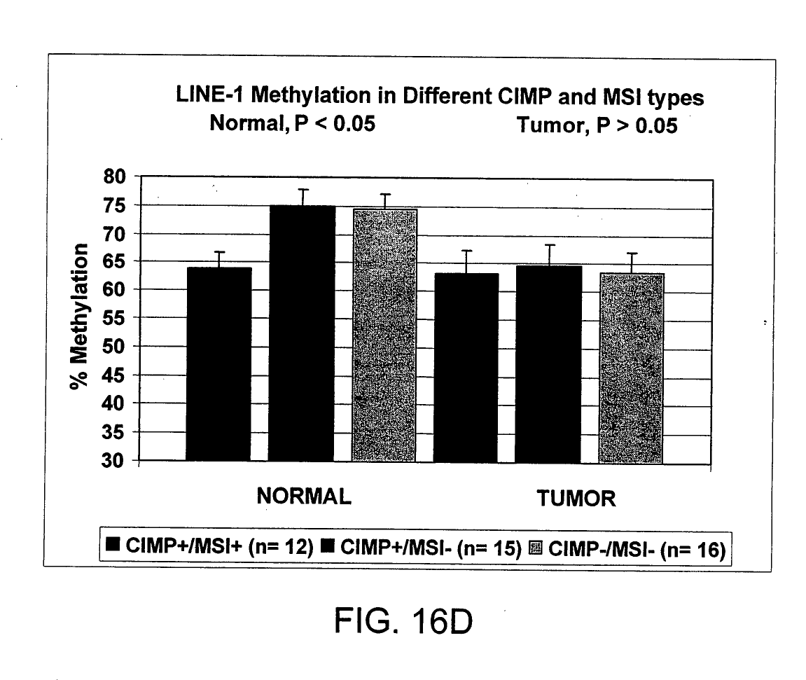

A total of 44 colorectal carcinomas (CRC) with their normal adjacent tissues were studied using the LINE-1 assay. Samples showed 64.1%±2.0% methylation compared to 72.0%±1.7% in normal adjacent tissue (P=0.003). LINE-1 methylation did not correlate with age, gender and tumor stage (FIG. 16A, B).

CRC with sporadic microsatellite instability (MSI) were studied, most of which are due to a CpG island methylation phenotype (CIMP) and associated MLH1 promoter methylation. In the CIMP+/MSI+ group, there was no difference in LINE-1 methylation between normal adjacent and cancer tissues (63.9%±2.8% versus 63.5%±2.4%, P=0.86), with a decrease in methylation of only 0.17%±3.8% (FIG. 16 C, D). In contrast with the CIMP+/MSI+ group, CIMP+/MSI− and CIMP− cases had similar decrease in LINE-1 methylation between normal adjacent and cancer tissues (15.1%±2.7% versus 16.5%±2.5%, P=0.71). This result could be partially due to the fact that LINE-1 methylation was lower in the normal adjacent tissue of the CIMP+/MSI+ group (63.9%±2.8%) compared to CIMP+/MSI− (75.0%±2.8%) and CIMP− (74.3%±2.7%) (P=0.008) groups (FIG. 16).

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Antequera, F., et al., DNA methylation in the fungi. Journal of Biological Chemistry, 1984. 259(13): p. 8033-6.
Belinsky, S. A., et al., Increased cytosine DNA-methyltransferase activity is target-cell-specific and an early event in lung cancer. Proceedings of the National Academy of Sciences of the United States of America., 1996. 93(9): p. 4045-50.
Bestor, T. H., S. B. Hellewell, and V. M. Ingram, Differentiation of two mouse cell lines is associated with hypomethylation of their genomes. Molecular & Cellular Biology, 1984. 4(9): p. 1800-6.
Bestor, T. H., The host defence function of genomic methylation patterns. Novartis Foundation symposium., 1998. 214.
Bird, A., The essentials of DNA methylation. Cell, 1992. 70(1): p. 5-8.
Clark, S. J., et al., High sensitivity mapping of methylated cytosines. Nucleic Acids Research, 1994. 22(15): p. 2990-7.
Feinberg, A. P. and B. Vogelstein, Hypomethylation distinguishes genes of some human cancers from their normal counterparts. Nature, 1983. 301(5895): p. 89-92.
Friso, S., et al., A common mutation in the 5,10-methylenetetrahydrofolate reductase gene affects genomic DNA methylation through an interaction with folate status. Proceedings of the National Academy of Sciences of the United States of America, 2002. 99(8): p. 5606-11.
Glover, A. B., et al., Azacitidine: 10 years later. Cancer Treatment Reports, 1987. 71(7-8): p. 737-46.
Gonzalgo, M. L., et al., Rapid quantitation of methylation differences at specific sites using methylation-sensitive single nucleotide primer extension (Ms-SNuPE). Nucleic acids research., 1997. 25(12): p. 2529-31.
Gu, Z., et al., Densities, length proportions, and other distributional features of repetitive sequences in the human genome estimatedfrom 430 megabases of genomic sequence. Gene, 2000. 259(1-2): p. 81-8.
Herman, J. G., et al., Methylation-specific PCR: a novel PCR assay for methylation status of CpG islands. Proceedings of the National Academy of Sciences of the United States of America., 1996. 93(18): p. 9821-6.
Hwu, H. R., et al., Insertion and/or deletion of many repeated DNA sequences in human and higher ape evolution. Proceedings of the National Academy of Sciences of the United States of America, 1986. 83(11): p. 3875-9.
Issa, J. P., CpG-island methylation in aging and cancer. Current Topics in Microbiology & Immunology, 2000. 249: p. 101-18.
Jones, P. A., et al., The role of DNA methylation in mammalian epigenetics. Science., 2001. 293(5532): p. 1068-70.
Jones, P. A. and S. B. Baylin, The fundamental role of epigenetic events in cancer. Nature Reviews Genetics, 2002. 3(6): p. 415-28.
Kazazian, H. H., Jr., J. L. Goodier, and S.o.M.C.R.B.C.B.U.o.P.P.U.S.A.k.m.m.u.e. Department of Genetics, LINE drive. retrotransposition and genome instability. Cell., 2002. 110(3): p. 277-80.
Kochanek, S., D. Renz, and W. Doerfler, DNA methylation in the Alu sequences of diploid and haploid primary human cells. EMBO Journal, 1993. 12(3): p. 1141-51.
Oakeley, E. J., DNA methylation analysis: a review of current methodologies. Pharmacology & therapeutics., 1999. 84(3): p. 389-400.
Richardson, B., Impact of aging on DNA methylation. Ageing research reviews., 2003. 2(3): p. 245-61.
Rideout, W. M., 3rd, et al., 5-Methylcytosine as an endogenous mutagen in the human LDL receptor and p53 genes. Science, 1990. 249(4974): p. 1288-90.
Robertson, K. D., et al., DNMT1 forms a complex with Rb, E2F1 and HDAC1 and represses transcription from E2F-responsive promoters. Nature Genetics, 2000. 25(3): p. 338-42.
Ronaghi, M., et al., Analyses of secondary structures in DNA by pyrosequencing. Analytical Biochemistry, 1999. 267(1): p. 65-71.
Santini, V., H. M. Kantarjian, and J. P. Issa, Changes in DNA methylation in neoplasia: pathophysiology and therapeutic implications. Annals of Internal Medicine, 2001. 134(7): p. 573-86.
Schmid, C. W., Does SINE evolution preclude Alu function? Nucleic Acids Research, 1998. 26(20): p. 4541-50.
Uhlmann, K., et al., Evaluation of a potential epigenetic biomarker by quantitative methyl-single nucleotide polymorphism analysis. Electrophoresis, 2002. 23(24): p. 4072-9.
Wagner, I. and I. Capesius, Determination of 5-methylcytosine from plant DNA by high-performance liquid chromatography. Biochimica et biophysica acta., 1981. 654(1): p. 52-6.
Xiong, Z. and P. W. Laird, COBRA: a sensitive and quantitative DNA methylation assay. Nucleic Adids Research, 1997. 25(12): p. 2532-4.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of determining the methylation status of one or more repetitive DNA elements of a DNA molecule comprising:
obtaining the DNA molecule; and
analyzing the methylation status of one or more of the repetitive DNA elements.

2. The method of claim 1, wherein the analyzing comprises reacting the DNA molecule with a modifying agent to convert unmethylated cytosine residues to uracil residues to provide a modified DNA molecule.

3. The method of claim 2, wherein the modifying agent comprises sodium bisulfite.

4. The method of claim 2, wherein the analyzing comprises amplifying one or more of the modified repetitive DNA elements.

5. The method of claim 4, further comprising the step of digesting the amplified one or more repetitive DNA elements with a restriction enzyme.

6. The method of claim 4, wherein the amplifying comprises using a forward consensus primer and a reverse consensus primer.

7. The method of claim 6, wherein the forward consensus primer and the reverse consensus primer comprise a restriction site at the 5-prime end.

8. The method of claim 7, wherein the restriction site is the MboI restriction site.

9. The method of claim 6, wherein the forward consensus primer comprises a linker sequence at the 5-prime end.

10. The method of claim 6, wherein the forward consensus primer comprises SEQ ID NO: 1 and the reverse consensus primer comprises SEQ ID NO:2.

11. The method of claim 6, wherein the forward consensus primer comprises SEQ ID NO:3 and the reverse consensus primer comprises SEQ ID NO:4.

12. The method of claim 6, wherein the forward consensus primer comprises SEQ ID NO:7 and the reverse consensus primer comprises SEQ ID NO:8.

13. The method of claim 1, wherein repetitive DNA elements comprise at least about 1,000 repetitive DNA elements.

14. The method of claim 1, wherein the repetitive DNA elements comprise at least about 10,000 repetitive DNA elements.

15. The method of claim 1, wherein the repetitive DNA element is a SINE element.

16. The method of claim 1, wherein the repetitive DNA element is an Alu element.

17. The method of claim 1, wherein the repetitive DNA element is a LINE element.

18. The method of claim 1, wherein the analyzing comprises Southern blotting, restriction digest analysis, or mass spectrometry.

19. The method of claim 1, wherein the analyzing comprises a sequencing reaction.

20. A method of monitoring efficacy of a methylation inhibiting drug in a patient comprising:
obtaining a DNA molecule with one or more repetitive DNA elements from a patient treated with a methylation inhibiting drug;
analyzing the methylation status of one or more of the repetitive DNA elements; and
comparing the methylation status of the one or more repetitive DNA elements to a control.

21. The method of claim 20, wherein the control is a DNA molecule taken from the patient before treatment with the methylation inhibiting drug.

22. The method of claim 20, wherein the methylation inhibiting drug is decitabine or 5-azacytidine.

23. The method of claim 20, wherein the methylation inhibiting drug is hydralazine or procainamide.

24. The method of claim 20, wherein the analyzing comprises reacting the DNA molecule with a modifying agent to convert unmethylated cytosine residues to uracil residues to provide a modified DNA molecule.

25. The method of claim 24, wherein the modifying agent comprises sodium bisulfite.

26. The method of claim 24, wherein the analyzing comprises amplifying one or more of the modified repetitive DNA elements.

27. The method of claim 26, further comprising the step of digesting the amplified one or more repetitive DNA elements with a restriction enzyme.

28. The method of claim 26, wherein the amplifying comprises using a forward consensus primer and a reverse consensus primer.

29. The method of claim 28, wherein the forward consensus primer and the reverse consensus primer comprise a restriction site at the 5-prime end.

30. The method of claim 29, wherein the restriction site is the MboI restriction site.

31. The method of claim 28, wherein the forward consensus primer comprises a linker sequence at the 5-prime end.

32. The method of claim 28, wherein the forward consensus primer comprises SEQ ID NO: 1 and the reverse consensus primer comprises SEQ ID NO:2.

33. The method of claim 28, wherein the forward consensus primer comprises SEQ ID NO:3 and the reverse consensus primer comprises SEQ ID NO:4.

34. The method of claim 28, wherein the forward consensus primer comprises SEQ ID NO:7 and the reverse consensus primer comprises SEQ ID NO:8.

35. The method of claim 20, wherein repetitive DNA elements comprise at least about 1,000 repetitive DNA elements.

36. The method of claim 20, wherein the repetitive DNA elements comprise at least about 10,000 repetitive DNA elements.

37. The method of claim 20, wherein the repetitive DNA element is a SINE element.

38. The method of claim 20, wherein the repetitive DNA element is an Alu element.

39. The method of claim 20, wherein the repetitive DNA element is a LINE element.

40. The method of claim 20, wherein the analyzing comprises Southern blotting, restriction digest analysis, or mass spectrometry.

41. The method of claim 20, wherein the analyzing comprises a sequencing reaction.

42. A method of diagnosing a disease associated with a change in methylation status comprising:
obtaining a DNA molecule with one or more repetitive DNA elements from a patient;
analyzing the methylation status of one or more of the repetitive DNA elements; and
comparing the methylation status of the one or more repetitive DNA elements to a control.

43. The method of claim 42, wherein disease associated with a change in methylation status is cancer, a genetic disorder, a metabolic disorder, a diseases associated with nutritional deficiency, or a disease associated with aging.

44. A primer comprising a consensus sequence of a modified DNA repetitive element.

45. A nucleic acid composition comprising SEQ ID NO: 1 and SEQ ID NO:2.

46. A nucleic acid composition comprising SEQ ID NO:3 and SEQ ID NO:4.

47. A nucleic acid composition comprising SEQ ID NO:7 and SEQ ID NO:8.

48. A kit for determining global methylation of a genomic DNA sample comprising at least one primer comprising a consensus sequence of a modified DNA repetitive element.
